(19) United States
(12) Patent Application Publication
(10) Pub. No.: US 2007/0238770 A1
(43) Pub. Date: Oct. 11, 2007

(54) PROCESS FOR PREPARING NOVEL CRYSTALLINE FORMS OF PELIGLITAZAR, NOVEL STABLE FORMS PRODUCED THEREIN AND FORMULATIONS

(75) Inventors: Jack Z. Gougoutas, Princeton, NJ (US); Yongmei Wu, East Brunswick, NJ (US); Soojin Kim, Demarest, NJ (US); Dejah T. Petsch, Ashford, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/695,124

(22) Filed: Apr. 2, 2007

Related U.S. Application Data
(60) Provisional application No. 60/789,382, filed on Apr. 5, 2006.

(51) Int. Cl. A61K 31/421; C07D 263/34

(52) U.S. Cl. 514/374; 548/236

(57) ABSTRACT

A process is provided for selectively preparing novel stable crystalline forms, namely selectively and consistently preparing Form N-1 of the free acid peliglitazar, Form N-2 of the free acid peliglitazar and Form P-1 of the L-lysine salt of the free acid peliglitazar. The process preferably employs solvent systems which produce crystals having suitable flow properties and desired particle size.

Novel Form N-1 crystals of the free acid, Form N-2 crystals of the free acid, Form P-1 crystals of the L-lysine salt of the free acid, pharmaceutical compositions containing such novel forms and a method of treating diabetes, dyslipidemia and atherosclerosis employing such novel forms are also provided.

### FIELD OF THE INVENTION

This application claims the benefit of U.S. provisional application Ser. No. 60/789,382 filed Apr. 5, 2006, the contents of which are herein incorporated by reference.

The present invention relates to a process for preparing novel stable crystalline forms, including Form N-1 of crystalline peliglitazar, Form N-2 of crystalline peliglitazar, and Form P-1 of the crystalline L-lysine salt of peliglitazar, to such novel Form N-1 and Form N-2 of crystalline peliglitazar, and Form P-1 of the crystalline L-lysine salt of peliglitazar, to pharmaceutical compositions containing such novel crystalline forms, and to methods of treating a mammal or diabetes, obesity and related conditions, including dyslipidemia, atherosclerosis and dysmetabolic syndrome employing such novel crystalline forms.

### BACKGROUND OF THE INVENTION

U.S. Pat. No. 6,414,002 to Cheng et al. discloses substituted acid derivatives which are useful as antidiabetic agents. One such compound disclosed is peliglitazar (Examples 498 and 498A) which has the structure I (also referred to herein as free acid I)

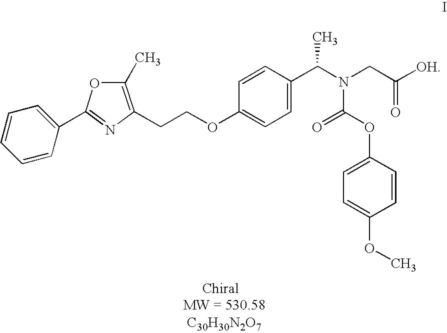

U.S. Pat. No. 6,414,002 discloses that the substituted acid derivatives of compounds of the invention form pharmaceutically acceptable salts such as alkali metal salts such as lithium, sodium or potassium, alkaline earth metal salts such as calcium or magnesium as well as zinc or aluminum and other cations such as ammonium, choline, diethanolamine, lysine (D or L), ethylenediamine, t-butylamine, t-octylamine, tris-(hydroxymethyl)aminomethane (TRIS), N-methyl glucosamine (NMG), triethanolamine and dehydroabietylamine.

Peliglitazar prepared in U.S. Pat. No. 6,414,002 is in amorphous or other non-crystalline form.

### BRIEF DESCRIPTION OF THE INVENTION

In accordance with the present invention, novel crystalline of forms of the free acid I including novel neat crystalline N-1 and N-2 forms and the crystalline L-lysine salt form of the free acid I and processes for preparing same are provided.

The free acid I (also referred to herein as peliglitazar) has the structure

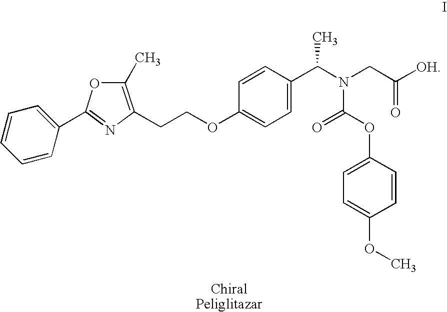

Preferred are the Form N-1 crystals of the free acid I which are non-hygroscopic with negligible moisture uptake at from 25 to 75% pH at 25° C., and can be isolated and remain stable in the solid state form. Form N-1 of the free acid I exhibits endotherm onset at about 123 to about 128° C. as observed by differential scanning calorimetry (DSC) and negligible weight loss up to about 110° C. as observed by thermogravimetric analysis (TGA).

The Form N-2 of the free acid I exhibits endotherm onset at about 130° C. as observed by DSC and negligible weight loss up to about 115° C.

Thermal analysis (DSC and TGA) indicates that Form N-1 of the free acid I and Form N-2 of the free acid I are enantiotropic polymorphs.

The Form P-1 of the L-lysine salt of free acid I has a solubility in water greater than 124 mg/mL (final pH 7.6) and exhibits endotherm onset at about 165° C. as observed from DSC and variable weight loss (0.3 to 1%) up to about 120° C. as observed from TGA.

The solid state stability of the free acid forms N-1 and N-2, as well as the P-1 form of the L-lysine salt as evaluated at room temperature/room light (RT/RL), 25° C./60% RH, 40° C./75% RH, 50° C. and high intensity light, DSC, powder x-ray diffraction (PXRD), and HPLC analysis indicate that all forms are stable (physically and chemically) for up to 2 weeks under the above conditions, except upon exposure to high intensity light. PXRD analysis shows that there is no conversion of the N-1 form to the N-2 form under the above conditions. No deliquescence is observed for any samples stored at 40° C./75% RH. Mechanical stress applied to the N-1 form will not cause conversion of N-1 form to the N-2 form.

In accordance with the present invention, a process is provided for preparing the crystalline Form N-1 free acid of peliglitazar, which includes the steps of

a) providing the free acid I (peliglitazar);

b) dissolving the free acid I in an organic solvent which can be methanol, ethanol, toluene, isopropyl alcohol, methanol/water, acetonitrile/water, N,N-dimethylacetamide (DMA), acetone, 2-butanone (MEK) or butyl acetate, preferably ethanol or isopropyl alcohol, preferably at a temperature within the range from about 40 to about 60° C., more preferably from about 45 to about 55° C., to obtain a solution;

c) seeding the solution from step b) with crystalline seeds of Form N-1 of the free acid I to initiate crystallization, the solution a) preferably being cooled to a temperature within the range from about 5 to about 25° C., preferably from about 15 to about 23° C., to form a slurry; and

d) recovering crystalline Form N-1 of the free acid I.

The seed crystals of Form N-1 of the free acid I may be prepared by recrystallizing or slurrying the crude free acid I in toluene, hexane or isopropyl alcohol.

In addition, in accordance with the present invention, a process is provided for preparing the crystalline Form N-2 of free acid I, which includes the steps of

a) providing the free acid I (peliglitazar);

b) dissolving the free acid I in isopropyl alcohol, preferably at a temperature within the range from about 45 to about 65° C., more preferably from about 55 to about 65° C., to obtain a solution;

c) seeding the solution from step b) with crystalline seeds of the Form N-2 of free acid I to initiate crystallization, the solution preferably being cooled to a temperature within the range from about 15 to about 23° C.; and

d) recovering crystalline N-2 free acid of peliglitazar.

In yet another embodiment of the invention, a process is provided for preparing the crystalline L-lysine salt of free acid I, which includes the steps of

a) providing the free acid I (peliglitazar);

b) dissolving the free acid I in ethanol, preferably at a temperature within the range from about 30 to about 50° C., more preferably from about 35 to about 45° C. to obtain a solution;

c) admixing L-lysine with the solution from step b) preferably at a temperature within the range from about 35 to about 65° C., more preferably from about 40 to about 60° C., to form a slurry;

d) cooling the slurry to a temperature within the range from about 5 to about 25° C., preferably from about 15 to about 20° C.; and

e) recovering the crystalline L-lysine salt of free acid I.

The Form N-1 and Form N-2 of the free acid I and the Form P-1 L-lysine salt of the free acid I, according to the invention, may be characterized using various techniques, the operation of which are well known to those of ordinary skill in the art. The forms may be characterized and distinguished using single crystal X-ray diffraction, which is based on unit cell measurements of a single crystal of a form at a fixed analytical temperature. A detailed description of unit cells is provided in Stout & Jensen, X-Ray Structure Determination: A Practical Guide, Macmillan Co., New York (1968), Chapter 3, which is herein incorporated by reference. Alternatively, the unique arrangement of atoms in spatial relation within the crystalline lattice may be characterized according to the observed fractional atomic coordinates. Another means of characterizing the crystalline structure is by powder X-ray diffraction analysis in which the experimental or observed diffraction profile is compared to a simulated profile representing pure powder material, both run at the same analytical temperature, and measurements for the subject form characterized as a series of 2θ values.

Other means of characterizing the form may be used, such as differential scanning calorimetry, thermogravimetric analysis, infrared spectra, moisture-sorption isotherms and hot-stage microscopy. These parameters may also be used in combination to characterize the subject form.

In one aspect of the present invention, Form N-1 of the free acid I may be characterized by unit cell parameters substantially equal to the following:

### Cell Dimensions:

a=10.387(3) Å

b=17.638(2) Å

c=15.073(4) Å

α=90°

β=96.70(2)°

γ=90°

Space group P2₁

Molecules/asymmetric unit 2

wherein the crystalline form is at about +22° C.

In a different aspect of the present invention, Form N-1 of the free acid I may be characterized by simulated and observed powder X-ray diffraction patterns as shown in FIG. 1.

In a different aspect of the present invention, Form N-1 of the free acid I may be characterized by fractional atomic coordinates substantially as listed in Table 2.

In a different aspect of the present invention, Form N-1 of the free acid I may be characterized by a powder X-ray diffraction (PXRD) pattern having the following 2θ values (CuKα λ=1.5418 Å) 5.9±0.1, 7.7±0.1, 10.0±0.1, 11.7±0.1, 12.8±0.1, 15.5±0.1, 16.2±0.1, 17.5±0.1, 19.2±0.1, 20.2±0.1.

In a different aspect of the present invention, Form N-1 of the free base I may be characterized by a differential scanning calorimetry (DSC) thermogram as shown in FIG. 4.

In a different aspect of the present invention, Form N-1 of the free base I may be characterized by a thermal gravimetric analysis (TGA) curve having a negligible weight loss up to about 110° C. as shown in FIG. 7.

In a different aspect of the present invention, Form N-1 of the free base I may be characterized by the moisture-sorption isotherm shown in FIG. 10.

In a different aspect of the present invention, Form N-1 of the free base I may be characterized by the Diamond ATR FT-IR spectrum shown in FIG. 11 and the selected unique IR-violational bonds (cm⁻¹): 1707, 1554, 1247, 1051, 856.

In another aspect of the present invention, Form N-2 of the free base I may be characterized by unit cell parameters substantially equal to the following:

### Single Crystal

### Cell Dimensions:

a=5.346(1) Å

b=20.75(2) Å

c=49.59(4) Å

α=90°

β=90°

γ=90°

ν(Å³)=5501(3)

Space group/orthorhombic

Molecules/asymmetric unit 2

wherein the crystalline form is at about +22° C.

In a different aspect of the present invention, Form N-2 of the free acid I may be characterized by simulated and observed powder X-ray diffraction patterns as shown in FIG. 2.

In a different aspect of the present invention, Form N-2 may be characterized by a powder X-ray diffraction pattern having the following 2θ values (CuKα λ=1.5418 Å) 3.6±0.1, 5.6±0.1, 6.8±0.1, 8.7±0.1, 12.3±0.1, 13.9±0.1, 14.9±0.1, 16.7±0.1, 17.1±0.1, 23.5±0.1.

In a different aspect of the present invention, Form N-2 of the free acid I may be characterized by a differential scanning calorimetry thermogram as shown in FIG. 5.

In a different aspect of the present invention, Form N-2 of the free acid I may be characterized by a thermal gravimetric analysis curve having a negligible weight loss up to about 115° C. as shown in FIG. 8.

In a different aspect of the present invention, Form N-2 of the free acid I may be characterized by the Diamond ATR FT-IR spectrum shown in FIG. 12.

In a different aspect of the present invention, Form P-1 of the L-lysine salt of the free acid I may be characterized by the observed powder X-ray diffraction patterns as shown in FIG. 3.

In a different aspect of the present invention, Form P-1 of the L-lysine salt of the free acid I may be characterized by a differential scanning calorimetry thermogram as shown in FIG. 6.

In a different aspect of the present invention, Form P-1 of the L-lysine salt of the free acid I may be characterized by a thermal gravimetric analysis curve having a negligible weight loss at about 120° C. as shown in FIG. 9.

The term “negligible weight loss”, as employed herein as characterized by TGA indicates the presence of a neat (non-solvated) crystal form.

The term “negligible % water uptake”, as employed herein, as characterized by a moisture-sorption isotherm indicates that the form tested is non-hygroscopic.

### BRIEF DESCRIPTION OF THE FIGURES

FIG. 1 shows calculated (simulated) (22° C.) and observed (experimental at room temperature) powder X-ray diffraction patterns (CuKα λ=1.5418 Å) of Form N-1 crystals of the free acid I (peliglitazar);

FIG. 2 shows calculated (simulated) (22° C.) and observed (RT) powder X-ray diffraction patterns (CuKα λ=1.5418 Å) of Form N-2 crystals of the free acid I;

FIG. 3 shows on observed (RT) powder X-ray diffraction pattern (CuKαλ=1.5418 Å) of Form P-1 crystals of the L-lysine salt of free acid I (peliglitazar);

FIG. 4 shows a differential scanning calorimetry (DSC) thermogram of Form N-1 crystals of the free acid I;

FIG. 5 shows a differential scanning calorimetry (DSC) thermogram of Form N-2 crystals of the free acid I;

FIG. 6 shows a differential scanning calorimetry (DSC) thermogram of Form P-1 crystals of the L-lysine salt of free acid I;

FIG. 7 shows a thermogravimetric analysis (TGA) curve of Form N-1 crystals of the free acid I;

FIG. 8 shows a thermogravimetric analysis (TGA) curve of Form N-2 crystals of the free acid I;

FIG. 9 shows a thermogravimetric analysis (TGA) curve of Form P-1 crystals of the L-lysine salt of the free acid I;

FIG. 10 is a moisture-sorption isotherm of Form N-1 crystals of the free acid I;

FIG. 11 is a Diamond ATR FT-IR spectrum of Form N-1 crystals of the free acid I; and

FIG. 12 is a Diamond ATR FT-IR spectrum of Form N-2 crystals of the free acid I.

### DETAILED DESCRIPTION OF THE INVENTION

The present invention provides, at least in part, crystalline forms of free acid I and salts thereof as a novel material, in particular in pharmaceutically acceptable form. The term “pharmaceutically acceptable”, as used herein, refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. In certain preferred embodiments, crystalline forms of free acid I, and salts thereof are in substantially pure form. The term “substantially pure”, as used herein, means a compound having a purity greater than about 90% including, for example, about 91%, about 92%, about 93%, about 94%, about 95%, about 96%, about 97%, about 98%, about 99%, and about 100%.

As used herein “polymorph” refers to crystalline forms having the same chemical composition but different spatial arrangements of the molecules, atoms, and/or ions forming the crystal.

As used herein “solvate” refers to a crystalline form of a molecule, atom, and/or ions that further contains molecules of a solvent or solvents incorporated into the crystalline structure. The solvent molecules in the solvate may be present in a regular arrangement and/or a non-ordered arrangement. The solvate may contain either a stoichiometric or nonstoichiometric amount of the solvent molecules. For example, a solvate with a nonstoichiometric amount of solvent molecules may result from partial loss of solvent from the solvate.

Samples of the crystalline forms may be provided with substantially pure phase homogeneity, indicating the presence of a dominant amount of a single crystalline form and optionally minor amounts of one or more other crystalline forms. The presence of more than one crystalline form in a sample may be determined by techniques such as powder X-ray diffraction (PXRD) or solid state nuclear magnetic resonance spectroscopy (SSNMR). For example, the presence of extra peaks in the comparison of an experimentally measured PXRD pattern with a simulated PXRD pattern may indicate more than one crystalline form in the sample. The simulated PXRD may be calculated from single crystal X-ray data. see Smith, D. K., “A FORTRAN Program for Calculating X-Ray Powder Diffraction Patterns,” Lawrence Radiation Laboratory, Livermore, Calif., UCRL-7196 (April 1963). Preferably, the crystalline form has substantially pure phase homogeneity as indicated by less than 10%, preferably less than 5%, and more preferably less than 2% of the total peak area in the experimentally measured PXRD pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

The crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture. High throughput crystallization techniques may be employed to prepare crystalline forms including polymorphs.

Crystals of drugs, including polymorphs, methods of preparation, and characterization of drug crystals are discussed in Solid-State Chemistry of Drugs, S. R. Byrn, R. R. Pfeiffer, and J. G. Stowell, 2^{nd} Edition, SSCI, West Lafayette, Ind. (1999).

For crystallization techniques that employ solvent, the choice of solvent or solvents is typically dependent upon one or more factors, such as solubility of the compound, crystallization technique, and vapor pressure of the solvent. Combinations of solvents may be employed, for example, the compound may be solubilized into a first solvent to afford a solution, followed by the addition of an antisolvent to decrease the solubility of the compound in the solution and to afford the formation of crystals. An antisolvent is a solvent in which the compound has low solubility. Suitable solvents for preparing crystals include polar and nonpolar solvents.

In one method to prepare crystals, free acid I or the L-lysine salt thereof is suspended and/or stirred in a suitable solvent to afford a slurry, which may be heated to promote dissolution. Suitable solvents in this regard include, for example, polar aprotic solvents, and polar protic solvents, and mixtures of two or more of these as disclosed herein.

The term “slurry”, as used herein, means a saturated solution of free acid I or L-lysine salt thereof, which may also contain an additional amount of free acid I or L-lysine salt thereof to afford a heterogeneous mixture of free acid I or L-lysine salt thereof and a solvent at a given temperature. Suitable solvents in this regard include, for example, polar aprotic solvents, and polar protic solvents, and mixtures of two or more of these as disclosed herein.

Seed crystals may be added to any crystallization mixture to promote crystallization. As will be clear to the skilled artisan, seeding is used as a means of controlling growth of a particular crystalline form or as a means of controlling the particle size distribution of the crystalline product. Accordingly, calculation of the amount of seeds needed depends on the size of the seed available and the desired size of an average product particle as described, for example, in “Programmed cooling of batch crystallizers,” J. W. Mullin and J. Nyvlt, Chemical Engineering Science (1971) 26:369-377. In general, seeds of small size are needed to effectively control the growth of crystals in the batch. Seeds of small size may be generated by sieving, milling, or micronizing of larger crystals, or by micro-crystallization of solutions. Care should be taken that milling or micronizing of crystals does not result in any change in crystallinity from the desired crystal form (i.e. change to amorphous or to another polymorph).

A cooled mixture may be filtered under vacuum, and the isolated solids may be washed with a suitable solvent, such as cold recrystallization solvent, and dried under a nitrogen purge to afford the desired crystalline form. The isolated solids may be analyzed by a suitable spectroscopic or analytical technique, such as SSNMR, DSC, PXRD, or the like, to assure formation of the preferred crystalline form of the product. The resulting crystalline form is typically produced in an amount of greater than about 70 weight % isolated yield, but preferably greater than 90 weight % based on the weight of free acid I originally employed in the crystallization procedure. The product may be comilled or passed through a mesh screen to delump the product, if necessary.

Crystalline forms may be prepared directly from the reaction medium of the final process step for preparing free acid I. This may be achieved, for example, by employing in the final process step a solvent or mixture of solvents from which free acid I may be crystallized. Alternatively, crystalline forms may be obtained by distillation or solvent addition techniques. Suitable solvents for this purpose include any of those solvents described herein, including protic polar solvents such as alcohols, and aprotic polar solvents such as ketones.

By way of general guidance, the reaction mixture may be filtered to remove any undesired impurities, inorganic salts, and the like, followed by washing with reaction or crystallization solvent. The resulting solution may be concentrated to remove excess solvent or gaseous constituents. If distillation is employed, the ultimate amount of distillate collected may vary, depending on process factors including, for example, vessel size, stirring capability, and the like. By way of general guidance, the reaction solution may be distilled to about 1/10 the original volume before solvent replacement is carried out. The reaction may be sampled and assayed to determine the extent of the reaction and the wt % product in accordance with standard process techniques. If desired, additional reaction solvent may be added or removed to optimize reaction concentration. Preferably, the final concentration is adjusted to about 50 wt % at which point a slurry typically results.

It may be preferable to add solvents directly to the reaction vessel without distilling the reaction mixture. Preferred solvents for this purpose are those which may ultimately participate in the crystalline lattice as discussed above in connection with solvent exchange. Although the final concentration may vary depending on desired purity, recovery and the like, the final concentration of free acid I in solution is preferably about 4% to about 7%. The reaction mixture may be stirred following solvent addition and simultaneously warmed. By way of illustration, the reaction mixture may be stirred for about 1 hour while warming to about 70° C. The reaction is preferably filtered hot and washed with either the reaction solvent, the solvent added or a combination thereof. Seed crystals may be added to any crystallization solution to initiate crystallization.

The various forms described herein may be distinguishable from one another through the use of various analytical techniques known to one of ordinary skill in the art. Such techniques include, but are not limited to, X-ray powder diffraction (PXRD), differential scanning calorimetry (DSC), thermogravimetric analysis (TGA), moisture-sorption isotherms, and/or IR spectrum.

One of ordinary skill in the art will appreciate that an X-ray diffraction pattern may be obtained with a measurement error that is dependent upon the measurement conditions employed. In particular, it is generally known that intensities in a X-ray diffraction pattern may fluctuate depending upon measurement conditions employed and the shape or morphology of the crystal. It should be further understood that relative intensities may also vary depending upon experimental conditions and, accordingly, the exact order of intensity should not be taken into account. Additionally, a measurement error of diffraction angle for a conventional X-ray diffraction pattern is typically about 0.2% or less, preferably about 0.1 (as discussed hereinafter), and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray diffraction patterns depicted in the accompanying Figures disclosed herein. Any crystal forms that provide X-ray diffraction patterns substantially identical to those disclosed in the accompanying Figures fall within the scope of the present invention. The ability to ascertain substantial identities of X-ray diffraction patterns is within the purview of one of ordinary skill in the art.

In carrying out a preferred process for preparing Form N-1 crystals of the free acid I, the free acid I is dissolved in an organic solvent such as ethanol, isopropyl alcohol, methanol, toluene, methanol/water, acetonitrile/water, N,N-dimethylacetamide (DMA), acetone, 2-butaneone (MEK) or butyl acetate, preferably ethanol or isopropyl alcohol, preferably at a temperature within the range from about 40 to about 60° C., more preferably from about 45 to about 55° C. to form a solution. The amount of free acid I employed will preferably be within the range from about 0.4 to about 3 g free acid I per 10 ml organic solvent, more preferably from about 0.5 to about 2.5 g free acid per 10 ml organic solvent.

The resulting solution is seeded with crystalline seeds of Form N-1 of the free acid I to initiate crystallization, employing an amount of seeds in a molar ratio of Form N-1 crystal seeds to free acid I within the range from about 0.00 1:1 to about 0.2:1, preferably from about 0.01:1 to about 0.05:1. The solution will thereby form a slurry which is cooled to a temperature within the range from about 25 to about 5° C., preferably from about 15 to about 23° C., and stirred for a period from about 1 to about 20 hours, preferably from about 5 to about 10 hours, filtered, washed with isopropyl alcohol or ethanol or other organic solvent as described above, and dried in vacuo to the Form N-1 crystals of the free acid I.

In carrying out the process for preparing seeds of Form N-1 crystals of the free acid I, the free acid I (which may be in amorphous form and prepared as described in U.S. Pat. No. 6,414,002, Example 498A) is dissolved in an organic solvent which is preferably hexane, heptane or hexane/ethyl acetate mixture, although other known organic solvents as set out above may be employed as well, as will be apparent to those skilled in the art. The amount of free acid I employed will be within the range from about 0.4 to about 2 g free acid per 10 ml of organic solvent, preferably from about 0.5 to about 1.5 g free acid per 10 ml of organic solvent.

In carrying out a preferred process for preparing Form N-2 crystals of the free acid I, the free acid I is dissolved in an organic solvent such as ethanol, isopropyl alcohol, methanol, toluene, methanol/water, acetonitrile/water, N,N-dimethylacetamide (DMA), acetone, 2-butaneone (MEK) or butyl acetate, preferably isopropyl alcohol, preferably at a temperature within the range from about 45 to about 65° C., more preferably from about 55 to about 65° C. to form a solution. The amount of free acid I employed will preferably be within the range from about 0.4 to about 3 g free acid I per 10 ml organic solvent, more preferably from about 0.5 to about 2.5 g free acid per 10 ml organic solvent.

The resulting solution is seeded with crystalline seeds of Form N-2 of the free acid I to initial crystallization, employing an amount of seeds in a molar ratio of Form N-2 crystal seeds to free acid I within the range from about 0.001:1 to about 0.2:1, preferably from about 0.01:1 to about 0.05:1. The solution will thereby form a slurry which is cooled to a temperature within the range from about 5 to about 25° C., preferably from about 15 to about 23° C., and stirred for a period from about 1 to about 20 hours, preferably from about 5 to about 10 hours, filtered, washed with isopropyl alcohol or ethanol or other organic solvent as described above, and dried in vacuo to the Form N-2 crystals of the free acid I.

In carrying out the process for preparing seeds of Form N-2 crystals of the free acid I, the free acid I is dissolved in an organic solvent which is preferably ethanol or isopropyl alcohol although other known organic solvents as set out above may be employed as well, as will be apparent to those skilled in the art. The amount of free acid I employed will be within the range from about 0.4 to about 2 g free acid per 10 ml of organic solvent, preferably from about 0.5 to about 1.5 g free acid per 10 ml of organic solvent.

In carrying out a preferred process for preparing Form P-1 crystals of the L-lysine salt of free acid I, the free acid I is dissolved in an organic solvent such as ethanol, isopropyl alcohol, methanol, toluene, methanol/water, acetonitrile/water, N,N-dimethylacetamide (DMA), acetone, 2-butaneone (MEK) or butyl acetate, preferably ethanol, preferably at a temperature within the range from about 30 to about 50° C., more preferably from about 35 to about 45° C. to form a solution. The amount of free acid I employed will preferably be within the range from about 0.4 to about 3 g free acid I per 10 ml organic solvent, more preferably from about 0.5 to about 2.5 g free acid per 10 ml organic solvent.

L-Lysine is mixed with the resulting solution employing an amount of a molar ratio of L-lysine to free acid I within the range from about 0.9:1 to about 2:1, preferably from about 1:1 to about 1.2:1. The solution will thereby form a slurry which is cooled to a temperature within the range from about 5 to about 25° C., preferably from about 15 to about 20° C., and stirred for a period from about 1 to about 20 hours, preferably from about 5 to about 10 hours, filtered, washed with isopropyl alcohol or ethanol or other organic solvent as described above, and dried in vacuo to the Form P-1 crystals of the L-lysine salt of the free acid I.

In addition, in accordance with the present invention, a method is provided for treating diabetes, especially Type 2 diabetes, and related diseases such as insulin resistance, hyperglycemia, hyperinsulinemia, dyslipidemia, elevated blood levels of fatty acids or glycerol, hyperlipidemia, obesity, hypertriglyceridemia, inflammation, diabetic complications, Syndrome X (dysmetabolic syndrome or metabolic syndrome), atherosclerosis, and related diseases wherein a therapeutically effective amount of Form N-1 of free acid I, Form N-2 of free acid I or Form P-1 of the L-lysine salt of free acid I is administered to a human patient in need of treatment.

In addition, in accordance with the present invention, a method is provided for treating early malignant lesions (such as ductal carcinoma in situ of the breast and lobular carcinoma in situ of the breast), premalignant lesions (such as fibroadenoma of the breast and prostatic intraepithelial neoplasia (PIN), liposarcomas and various other epithelial tumors (including breast, prostate, colon, ovarian, gastric and lung), irritable bowel syndrome, Crohn's disease, gastric ulceritis, and osteoporosis and proliferative diseases such as psoriasis, wherein a therapeutically effective amount of Form N-1 of free acid I, Form N-2 of free acid I or Form P-1 of the L-lysine salt of free acid I is administered to a human patient in need of treatment.

In addition, in accordance with the present invention, a method is provided for treating diabetes and related diseases as defined above and hereinafter, wherein a therapeutically effective amount of a combination of Form N-1 of free acid I, Form N-2 of free acid I or Form P-1 of the L-lysine salt of free acid I, and another type antidiabetic agent and/or a hypolipidemic agent, and/or lipid modulating agent and/or other type of therapeutic agent, is administered to a human patient in need of treatment.

In the above methods of the invention, the Form N-1 of free acid I, Form N-2 of free acid I or Form P-1 of the L-lysine salt of free acid I will be employed in a weight ratio to the antidiabetic agent (depending upon its mode of operation) within the range from about 0.01:1 to about 100:1, preferably from about 0.5:1 to about 10:1.

The conditions, diseases, and maladies collectively referenced to as “Syndrome X” or Dysmetabolic Syndrome or Metabolic Syndrome are detailed in Johannsson, J. Clin. Endocrinol. Metab., 82:727-734 (1997) and other publications.

The term “diabetes and related diseases” refers to Type II diabetes, Type I diabetes, impaired glucose tolerance, obesity, hyperglycemia, Syndrome X, dysmetabolic syndrome, diabetic complications and hyperinsulinemia.

The conditions, diseases and maladies collectively referred to as “diabetic complications” include retinopathy, neuropathy and nephropathy, and other known complications of diabetes.

The term “other type(s) of therapeutic agents” as employed herein refers to one or more antidiabetic agents (other than peliglitazar), one or more anti-obesity agents, and/or one or more lipid-lowering agents, one or more lipid modulating agents (including anti-atherosclerosis agents), and/or one or more antiplatelet agents, one or more agents for treating hypertension, one or more anti-cancer drugs, one or more agents for treating arthritis, one or more anti-osteoporosis agents, one or more anti-obesity agents, one or more agents for treating immunomodulatory diseases, and/or one or more agents for treating anorexia nervosa.

The term “lipid-modulating” agent as employed herein refers to agents which lower LDL and/or raise HDL and/or lower triglycerides and/or lower total cholesterol and/or other known mechanisms for therapeutically treating lipid disorders.

Where desired, the Form N-1 of free acid I, Form N-2 of free acid I or Form P-1 of the L-lysine salt of free acid I may be used in combination with one or more hypolipidemic agents or lipid-lowering agents and/or one or more other types of therapeutic agents including antidiabetic agents, anti-obesity agents, antihypertensive agents, platelet aggregation inhibitors, and/or anti-osteoporosis agents, which may be administered orally in the same dosage form, in a separate oral dosage form or by injection.

The hypolipidemic agent or lipid-lowering agent which may be optionally employed in combination with the Form N-1 of free acid I, Form N-2 of free acid I or Form P-1 of the L-lysine salt of free acid I may include 1, 2, 3 or more MTP inhibitors, HMG CoA reductase inhibitors, squalene synthetase inhibitors, fibric acid derivatives, ACAT inhibitors, lipoxygenase inhibitors, cholesterol absorption inhibitors, ileal Na⁺/bile acid cotransporter inhibitors, upregulators of LDL receptor activity, bile acid sequestrants, and/or nicotinic acid and derivatives thereof.

MTP inhibitors employed herein include MTP inhibitors disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. application Ser. No. 09/175,180 filed Oct. 20, 1998, now U.S. Pat. No. 5,962,440. Preferred are each of the preferred MTP inhibitors disclosed in each of the above patents and applications.

All of the above U.S. Patents and applications are incorporated herein by reference.

Most preferred MTP inhibitors to be employed in accordance with the present invention include preferred MTP inhibitors as set out in U.S. Pat. Nos. 5,739,135 and 5,712,279, and U.S. Pat. No. 5,760,246.

The most preferred MTP inhibitor is 9-[4-[4-[[2-(2,2,2-Trifluoroethoxy)benzoyl]amino]-1-piperidinyl]butyl]-N-(2,2,2-trifluoroethyl)-9H-fluorene-9-carboxamide

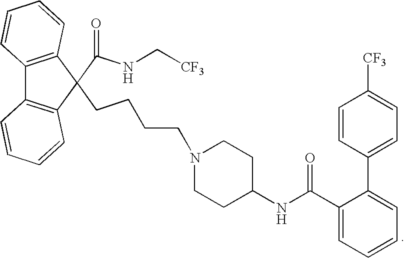

The hypolipidemic agent may be an HMG CoA reductase inhibitor which includes, but is not limited to, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, itavastatin (Nissan/Sankyo's nisvastatin (NK-104)) disclosed in U.S. Pat. No. 5,011,930, Shionogi-Astra/Zeneca visastatin (ZD-4522) disclosed in U.S. Pat. No. 5,260,440, and related statin compounds disclosed in U.S. Pat. No. 5,753,675, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, and quinoline and pyridine derivatives disclosed in U.S. Pat. Nos. 5,506,219 and 5,691,322.

In addition, phosphinic acid compounds useful in inhibiting HMG CoA reductase suitable for use herein are disclosed in GB 2205837.

The squalene synthetase inhibitors suitable for use herein include, but are not limited to, α-phosphono-sulfonates disclosed in U.S. Pat. No. 5,712,396, those disclosed by Biller et al., J. Med. Chem., 1988, Vol. 31, No. 10, pp 1869-1871, including isoprenoid (phosphinyl-methyl)phosphonates as well as other known squalene synthetase inhibitors, for example, as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996).

In addition, other squalene synthetase inhibitors suitable for use herein include the terpenoid pyrophosphates disclosed by P. Ortiz de Montellano et al., J. Med. Chem., 1977, 20:243-249, the farnesyl diphosphate analog A and presqualene pyrophosphate (PSQ-PP) analogs as disclosed by Corey and Volante, J. Am. Chem. Soc., 1976, 98:1291-1293, phosphinylphosphonates reported by McClard, R. W. et al., J.A.C.S., 1987, 109:5544 and cyclopropanes reported by Capson, T. L., PhD dissertation, June, 1987, Dept. Med. Chem. U of Utah, Abstract, Table of Contents, pp 16, 17, 40-43, 48-51, Summary.

Other hypolipidemic agents suitable for use herein include, but are not limited to, fibric acid derivatives, such as fenofibrate, gemfibrozil, clofibrate, bezafibrate, ciprofibrate, clinofibrate and the like, probucol, and related compounds as disclosed in U.S. Pat. No. 3,674,836, probucol and gemfibrozil being preferred, bile acid sequestrants such as cholestyramine, colestipol and DEAE-Sephadex (Secholex®, Policexide®) and cholestagel (Sankyo/Geltex), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid (niacin), acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride) and ionenes such as disclosed in U.S. Pat. No. 4,027,009, and other known serum cholesterol lowering agents.

The hypolipidemic agent may be an ACAT inhibitor such as disclosed in, Drugs of the Future 24:9-15 (1999), (Avasimibe); “The ACAT inhibitor, C1-1011 is effective in the prevention and regression of aortic fatty streak area in hamsters”, Nicolosi et al., Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al., Bioorg. Med. Chem. Lett. (1996), 6(1), 47-50; “ACAT inhibitors: physiologic mechanisms for hypolipidemic and anti-atherosclerotic activities in experimental animals”, Krause et al., Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al., Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al., Chemtracts: Org. Chem. (1995), 8(6), 359-62, or TS-962 (Taisho Pharmaceutical Co. Ltd).

The hypolipidemic agent may be an upregulator of LD2 receptor activity such as MD-700 (Taisho Pharmaceutical Co. Ltd) and LY295427 (Eli Lilly).

The hypolipidemic agent may be a cholesterol absorption inhibitor preferably Schering-Plough's SCH48461 as well as those disclosed in Atherosclerosis 115:45-63 (1995) and J. Med. Chem. 41:973 (1998).

The hypolipidemic agent may be an ileal Na⁺/bile acid cotransporter inhibitor such as disclosed in Drugs of the Future, 24:425-430 (1999).

The lipid-modulating agent may be a cholesteryl ester transfer protein (CETP) inhibitor such as Pfizer's CP 529,414 (WO/0038722 and EP 818448) and Pharmacia's SC-744 and SC-795.

The ATP citrate lyase inhibitor which may be employed in the combination of the invention may include, for example, those disclosed in U.S. Pat. No. 5,447,954.

Preferred hypolipidemic agents are pravastatin, lovastatin, simvastatin, atorvastatin, fluvastatin, cerivastatin, itavastatin and visastatin and ZD-4522.

The above-mentioned U.S. patents are incorporated herein by reference. The amounts and dosages employed will be as indicated in the Physician's Desk Reference and/or in the patents set out above.

The Form N-1 of free acid I, Form N-2 of free acid I, and Form P-1 of the L-lysine salt of free acid I compounds of formula I of the invention will be employed in a weight ratio to the hypolipidemic agent (were present), within the range from about 500:1 to about 1:500, preferably from about 100:1 to about 1:100.

The dose administered must be carefully adjusted according to age, weight and condition of the patient, as well as the route of administration, dosage form and regimen and the desired result.

The dosages and formulations for the hypolipidemic agent will be as disclosed in the various patents and applications discussed above.

The dosages and formulations for the other hypolipidemic agent to be employed, where applicable, will be as set out in the latest edition of the Physicians' Desk Reference.

For oral administration, a satisfactory result may be obtained employing the MTP inhibitor in an amount within the range of from about 0.01 mg to about 500 mg and preferably from about 0.1 mg to about 100 mg, one to four times daily.

A preferred oral dosage form, such as tablets or capsules, will contain the MTP inhibitor in an amount of from about 1 to about 500 mg, preferably from about 2 to about 400 mg, and more preferably from about 5 to about 250 mg, one to four times daily.

For oral administration, a satisfactory result may be obtained employing an HMG CoA reductase inhibitor, for example, pravastatin, lovastatin, simvastatin, atorvastatin, fluvastatin or cerivastatin in dosages employed as indicated in the Physician's Desk Reference, such as in an amount within the range of from about 1 to 2000 mg, and preferably from about 4 to about 200 mg.

The squalene synthetase inhibitor may be employed in dosages in an amount within the range of from about 10 mg to about 2000 mg and preferably from about 25 mg to about 200 mg.

A preferred oral dosage form, such as tablets or capsules, will contain the HMG CoA reductase inhibitor in an amount from about 0.1 to about 100 mg, preferably from about 0.5 to about 80 mg, and more preferably from about 1 to about 40 mg.

A preferred oral dosage form, such as tablets or capsules will contain the squalene synthetase inhibitor in an amount of from about 10 to about 500 mg, preferably from about 25 to about 200 mg.

The hypolipidemic agent may also be a lipoxygenase inhibitor including a 15-lipoxygenase (15-LO) inhibitor such as benzimidazole derivatives as disclosed in WO 97/12615, 15-LO inhibitors as disclosed in WO 97/12613, isothiazolones as disclosed in WO 96/38144, and 15-LO inhibitors as disclosed by Sendobry et al. “Attenuation of diet-induced atherosclerosis in rabbits with a highly selective 15-lipoxygenase inhibitor lacking significant antioxidant properties”, Brit. J. Pharmacology (1997) 120:1199-1206, and Cornicelli et al., “15-Lipoxygenase and its Inhibition: A Novel Therapeutic Target for Vascular Disease”, Current Pharmaceutical Design, 1999, 5:11-20.

The Form N-1 of free acid I, Form N-2 of free acid I, and Form P-1 of the L-lysine salt of free acid I and the hypolipidemic agent may be employed together in the same oral dosage form or in separate oral dosage forms taken at the same time.

The compositions described above may be administered in the dosage forms as described above in single or divided doses of one to four times daily. It may be advisable to start a patient on a low dose combination and work up gradually to a high dose combination.

The preferred hypolipidemic agent is pravastatin, simvastatin, lovastatin, atorvastatin, fluvastatin or cerivastatin as well as niacin and/or cholestagel.

The other antidiabetic agent which may be optionally employed in combination with the Form N-1 of free acid I, Form N-2 of free acid I, and Form P-1 of the L-lysine salt of free acid I may be 1, 2, 3 or more antidiabetic agents or antihyperglycemic agents including insulin secretagogues or insulin sensitizers, or other antidiabetic agents preferably having a mechanism of action different from the compounds of formula I of the invention, which may include biguanides, sulfonyl ureas, glucosidase inhibitors, PPAR γ agonists, such as thiazolidinediones, aP2 inhibitors, dipeptidyl peptidase IV (DP4) inhibitors, SGLT2 inhibitors, and/or meglitinides, as well as insulin, and/or glucagon-like peptide-1 (GLP-1).

The other antidiabetic agent may be an oral antihyperglycemic agent preferably a biguanide such as metformin or phenformin or salts thereof, preferably metformin HCl.

Where the antidiabetic agent is a biguanide, the Form N-1 of free acid I, Form N-2 of free acid I, and Form P-1 of the L-lysine salt of free acid I will be employed in a weight ratio to biguanide within the range from about 0.001:1 to about 10:1, preferably from about 0.01:1 to about 5:1.

The other antidiabetic agent may also preferably be a sulfonyl urea such as glyburide (also known as glibenclamide), glimepiride (disclosed in U.S. Pat. No. 4,379,785), glipizide, gliclazide or chlorpropamide, other known sulfonylureas or other antihyperglycemic agents which act on the ATP-dependent channel of the β-cells, with glyburide and glipizide being preferred, which may be administered in the same or in separate oral dosage forms.

The Form N-1 of free acid I, Form N-2 of free acid I, and Form P-1 of the L-lysine salt of free acid I will be employed in a weight ratio to the sulfonyl urea in the range from about 0.01:1 to about 100:1, preferably from about 0.02:1 to about 5:1.

The oral antidiabetic agent may also be a glucosidase inhibitor such as acarbose (disclosed in U.S. Pat. No. 4,904,769) or miglitol (disclosed in U.S. Pat. No. 4,639,436), which may be administered in the same or in a separate oral dosage forms.

The Form N-1 of free acid I, Form N-2 of free acid I, and Form P-1 of the L-lysine salt of free acid I will be employed in a weight ratio to the glucosidase inhibitor within the range from about 0.01:1 to about 100:1, preferably from about 0.05:1 to about 10:1.

The Form N-1 of free acid I, Form N-2 of free acid I, and Form P-1 of the L-lysine salt of free acid I may be employed in combination with a PPAR γ agonist such as a thiazolidinedione oral anti-diabetic agent or other insulin sensitizers (which has an insulin sensitivity effect in NIDDM patients) such as troglitazone (Warner-Lambert's Rezulin®, disclosed in U.S. Pat. No. 4,572,912), rosiglitazone (SKB), pioglitazone (Takeda), Mitsubishi's MCC-555 (disclosed in U.S. Pat. No. 5,594,016), Glaxo-Wellcome's GL-262570, englitazone (CP-68722, Pfizer) or darglitazone (CP-86325), Pfizer, isaglitazone (MIT/J&J), JTT-501 (JPNT/P&U), L-895645 (Merck), R-119702 (Sankyo/WL), NN-2344 (Dr. Reddy/NN), or YM-440 (Yamanouchi), preferably rosiglitazone and pioglitazone, and/or LDL lowering agents such as torcetrapid, ezetimibe, a combination of atorvastatin and torcetrapid, or a combination of simvastatin and ezetimibe.

The Form N-1 of free acid I, Form N-2 of free acid I, and Form P-1 of the L-lysine salt of free acid I will be employed in a weight ratio to the thiazolidinedione in an amount within the range from about 0.01:1 to about 100:1, preferably from about 0.05 to about 10:1.

The sulfonyl urea and thiazolidinedione in amounts of less than about 150 mg oral antidiabetic agent may be incorporated in a single tablet with the Form N-1 of free acid I, Form N-2 of free acid I, and Form P-1 of the L-lysine salt of free acid I.

The Form N-1 of free acid I, Form N-2 of free acid I, and Form P-1 of the L-lysine salt of free acid I may also be employed in combination with a antihyperglycemic agent such as insulin or with glucagon-like peptide-1 (GLP-1) such as GLP-1(1-36) amide, GLP-1(7-36) amide, GLP-1(7-37) (as disclosed in U.S. Pat. No. 5,614,492 to Habener, the disclosure of which is incorporated herein by reference), as well as AC2993 (Amylin) and LY-315902 (Lilly), which may be administered via injection, intranasal, inhalation or by transdermal or buccal devices.

Where present, metformin, the sulfonyl ureas, such as glyburide, glimepiride, glipyride, glipizide, chlorpropamide and gliclazide and the glucosidase inhibitors acarbose or miglitol or insulin (injectable, pulmonary, buccal, or oral) may be employed in formulations as described above and in amounts and dosing as indicated in the Physician's Desk Reference (PDR).

Where present, metformin or salt thereof may be employed in amounts within the range from about 500 to about 2000 mg per day which may be administered in single or divided doses one to four times daily.

Where present, the thiazolidinedione anti-diabetic agent may be employed in amounts within the range from about 0.01 to about 2000 mg/day which may be administered in single or divided doses one to four times per day.

Where present insulin may be employed in formulations, amounts and dosing as indicated by the Physician's Desk Reference.

Where present GLP-1 peptides may be administered in oral buccal formulations, by nasal administration or parenterally as described in U.S. Pat. Nos. 5,346,701 (TheraTech), 5,614,492 and 5,631,224 which are incorporated herein by reference.

The other antidiabetic agent may also be a PPAR α/γ dual agonist such as AR-HO39242 (Astra/Zeneca), GW-409544 (Glaxo-Wellcome), KRP297 (Kyorin Merck) as well as those disclosed by Murakami et al., “A Novel Insulin Sensitizer Acts As a Coligand for Peroxisome Proliferation—Activated Receptor Alpha (PPAR alpha) and PPAR gamma. Effect on PPAR alpha Activation on Abnormal Lipid Metabolism in Liver of Zucker Fatty Rats”, Diabetes 47:1841-1847 (1998).

The antidiabetic agent may be an SGLT2 inhibitor such as disclosed in U.S. provisional application No. 60/158,773, filed Oct. 12, 1999 (attorney file LA49), now U.S. Pat. No. 6,414,126 employing dosages as set out therein. Preferred are the compounds designated as preferred in the above application.

The antidiabetic agent may be an aP2 inhibitor such as disclosed in U.S. application Ser. No. 09/391,053, filed Sep. 7, 1999, and in U.S. provisional application No. 60/127,745, filed Apr. 5, 1999 (attorney file LA27*), now U.S. Pat. No. 6,548,529 employing dosages as set out herein. Preferred are the compounds designated as preferred in the above application.

The antidiabetic agent may be a DP4 inhibitor such as disclosed in Provisional Application 60/188,555 filed Mar. 10, 2000 (attorney file LA50), now U.S. Pat. No. 6,395,767, WO99/38501, WO99/46272, WO99/67279 (PROBIODRUG), WO99/67278 (PROBIODRUG), WO99/61431 (PROBIODRUG), NVP-DPP728A (1-[[[2-[(5-cyanopyridin-2-yl)amino]ethyl]amino]acetyl]-2-cyano-(S)-pyrrolidine) (Novartis) (preferred) as disclosed by Hughes et al., Biochemistry, 38(36), 11597-11603, 1999, TSL-225 (tryptophyl-1,2,3,4-tetrahydro-isoquinoline-3-carboxylic acid (disclosed by Yamada et al., Bioorg. & Med. Chem. Lett. 8 (1998) 1537-1540, 2-cyanopyrrolidides and 4-cyanopyrrolidides as disclosed by Ashworth et al., Bioorg. & Med. Chem. Lett., Vol. 6, No. 22, pp 1163-1166 and 2745-2748 (1996) employing dosages as set out in the above references.

The meglitinide which may optionally be employed in combination with the Form N-1 of free acid I, Form N-2 of free acid I, and Form P-1 of the L-lysine salt of free acid I of the invention may be repaglinide, nateglinide (Novartis) or KAD1229 (PF/Kissei), with repaglinide being preferred.

The Form N-1 of free acid I, Form N-2 of free acid I, and Form P-1 of the L-lysine salt of free acid I will be employed in a weight ratio to the meglitinide, PPAR γ agonist, PPAR α/γ dual agonist, aP2 inhibitor, DP4 inhibitor or SGLT2 inhibitor within the range from about 0.01:1 to about 100:1, preferably from about 0.05 to about 10:1.

The other type of therapeutic agent which may be optionally employed with Form N-1 of free acid I, Form N-2 of free acid I, and Form P-1 of the L-lysine salt of free acid I may be 1, 2, 3 or more of an anti-obesity agent including a beta 3 adrenergic agonist, a lipase inhibitor, a serotonin (and dopamine) reuptake inhibitor, an aP2 inhibitor, a thyroid receptor agonist and/or an anorectic agent.

The beta 3 adrenergic agonist which may be optionally employed in combination with Form N-1 of free acid I, Form N-2 of free acid I, and Form P-1 of the L-lysine salt of free acid I may be AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, with AJ9677, L750,355 and CP331648 being preferred.

The lipase inhibitor which may be optionally employed in combination with Form N-1 of free acid I, Form N-2 of free acid I, and Form P-1 of the L-lysine salt of free acid I may be orlistat or ATL-962 (Alizyme), with orlistat being preferred.

The serotonin (and dopoamine) reuptake inhibitor which may be optionally employed in combination with Form N-1 of free acid I, Form N-2 of free acid I, and Form P-1 of the L-lysine salt of free acid I may be sibutramine, topiramate (Johnson & Johnson) or axokine (Regeneron), with sibutramine and topiramate being preferred.

The thyroid receptor agonist which may be optionally employed in combination with Form N-1 of free acid I, Form N-2 of free acid I, and Form P-1 of the L-lysine salt of free acid I may be a thyroid receptor ligand as disclosed in WO97/21993 (U. Cal SF), WO99/00353 (KaroBio), WO00/039077 (KaroBio), and U.S. Provisional Application 60/183,223 filed Feb. 17, 2000, with compounds of the KaroBio applications and the above U.S. provisional application being preferred.

The anorectic agent which may be optionally employed in combination with Form N-1 of free acid I, Form N-2 of free acid I, and Form P-1 of the L-lysine salt of free acid I may be dexamphetamine, phentermine, phenylpropanolamine or mazindol, with dexamphetamine being preferred.

The various anti-obesity agents described above may be employed in the same dosage form with Form N-1 of free acid I, Form N-2 of free acid I, and Form P-1 of the L-lysine salt of free acid I or in different dosage forms, in dosages and regimens as generally known in the art or in the PDR.

The antihypertensive agents which may be employed in combination with Form N-1 of free acid I, Form N-2 of free acid I, and Form P-1 of the L-lysine salt of free acid I of the invention include ACE inhibitors, angiotensin II receptor antagonists, NEP/ACE inhibitors, as well as calcium channel blockers, β-adrenergic blockers and other types of antihypertensive agents including diuretics.

The angiotensin converting enzyme inhibitor which may be employed herein includes those containing a mercapto (—S—) moiety such as substituted proline derivatives, such as any of those disclosed in U.S. Pat. No. 4,046,889 to Ondetti et al. mentioned above, with captopril, that is, 1-[(2S)-3-mercapto-2-methylpropionyl]-L-proline, being preferred, and mercaptoacyl derivatives of substituted prolines such as any of those disclosed in U.S. Pat. No. 4,316,906 with zofenopril being preferred.

Other examples of mercapto containing ACE inhibitors that may be employed herein include rentiapril (fentiapril, Santen) disclosed in Clin. Exp. Pharmacol. Physiol. 10:131 (1983); as well as pivopril and YS980.

Other examples of angiotensin converting enzyme inhibitors which may be employed herein include any of those disclosed in U.S. Pat. No. 4,374,829 mentioned above, with N-(1-ethoxycarbonyl-3-phenylpropyl)-L-alanyl-L-proline, that is, enalapril, being preferred, any of the phosphonate substituted amino or imino acids or salts disclosed in U.S. Pat. No. 4,452,790 with (S)-1-[6-amino-2-[[hydroxy-(4-phenylbutyl)phosphinyl]oxy]-1-oxohexyl]-L-proline or (ceronapril) being preferred, phosphinylalkanoyl prolines disclosed in U.S. Pat. No. 4,168,267 mentioned above with fosinopril being preferred, any of the phosphinylalkanoyl substituted prolines disclosed in U.S. Pat. No. 4,337,201, and the phosphonamidates disclosed in U.S. Pat. No. 4,432,971 discussed above.

Other examples of ACE inhibitors that may be employed herein include Beecham's BRL 36,378 as disclosed in European Patent Application Nos. 80822 and 60668; Chugai's MC-838 disclosed in C.A. 102:72588v and Jap. J. Pharmacol. 40:373 (1986); Ciba-Geigy's CGS 14824 (3-([1-ethoxycarbonyl-3-phenyl-(1S)-propyl]amino)-2,3,4,5-tetrahydro-2-oxo-1-(3S)-benzazepine-1 acetic acid HCl) disclosed in U.K. Patent No. 2103614 and CGS 16,617 (3(S)-[[(1S)-5-amino-1-carboxypentyl]amino]-2,3,4,5-tetrahydro-2-oxo-1H-1-benzazepine-1-ethanoic acid) disclosed in U.S. Pat. No. 4,473,575; cetapril (alacepril, Dainippon) disclosed in Eur. Therap. Res. 39:671 (1986); 40:543 (1986); ramipril (Hoechsst) disclosed in Euro. Patent No. 79-022 and Curr. Ther. Res. 40:74 (1986); Ru 44570 (Hoechst) disclosed in Arzneimittelforschung 34:1254 (1985), cilazapril (Hoffman-LaRoche) disclosed in J. Cardiovasc. Pharmacol. 9:39 (1987); R 31-2201 (Hoffman-LaRoche) disclosed in FEBS Lett. 165:201 (1984); lisinopril (Merck), indalapril (delapril) disclosed in U.S. Pat. No. 4,385,051; indolapril (Schering) disclosed in J. Cardiovasc. Pharmacol. 5:643, 655 (1983), spirapril (Schering) disclosed in Acta. Pharmacol. Toxicol. 59 (Supp. 5):173 (1986); perindopril (Servier) disclosed in Eur. J. Clin. Pharmacol. 31:519 (1987); quinapril (Warner-Lambert) disclosed in U.S. Pat. No. 4,344,949 and CI925 (Warner-Lambert) ([3 S-[2 [R(*)R(*)]]3R(*)]-2-[2-[[1-(ethoxy-carbonyl)-3-phenylpropyl]amino]-1-oxopropyl]-1,2,3,4-tetrahydro-6,7-dimethoxy-3-isoquinolinecarboxylic acid HCl)disclosed in Pharmacologist 26:243, 266 (1984), WY-44221 (Wyeth) disclosed in J. Med. Chem. 26:394 (1983).

Preferred ACE inhibitors are captopril, fosinopril, enalapril, lisinopril, quinapril, benazepril, fentiapril, ramipril and moexipril.

NEP/ACE inhibitors may also be employed herein in that they possess neutral endopeptidase (NEP) inhibitory activity and angiotensin converting enzyme (ACE) inhibitory activity. Examples of NEP/ACE inhibitors suitable for use herein include those disclosed in U.S. Pat. Nos. 5,362,727, 5,366,973, 5,225,401, 4,722,810, 5,223,516, 4,749,688, U.S. Pat. No. 5,552,397, U.S. Pat. No. 5,504,080, U.S. Pat. No. 5,612,359,U.S. Pat. No. 5,525,723, European Patent Application 0599,444, 0481,522, 0599,444, 0595,610, European Patent Application 0534363A2, 534,396 and 534,492, and European Patent Application 0629627A2.

Preferred are those NEP/ACE inhibitors and dosages thereof which are designated as preferred in the above patents/applications which U.S. patents are incorporated herein by reference; most preferred are omapatrilat, BMS 189,921 ([S—(R*,R*)]-hexahydro-6-[(2-mercapto-1-oxo-3-phenylpropyl)amino]-2,2-dimethyl-7-oxo-1H-azepine-1-acetic acid (gemopatrilat)) and CGS 30440.

The angiotensin II receptor antagonist (also referred to herein as angiotensin II antagonist or AII antagonist) suitable for use herein includes, but is not limited to, irbesartan, losartan, valsartan, candesartan, telmisartan, tasosartan or eprosartan, with irbesartan, losartan or valsartan being preferred.

A preferred oral dosage form, such as tablets or capsules, will contain the ACE inhibitor or AII antagonist in an amount within the range from abut 0.1 to about 500 mg, preferably from about 5 to about 200 mg and more preferably from about 10 to about 150 mg.

For parenteral administration, the ACE inhibitor, angiotensin II antagonist or NEP/ACE inhibitor will be employed in an amount within the range from about 0.005 mg/kg to about 10 mg/kg and preferably from about 0.01 mg/kg to about 1 mg/kg.

Where a drug is to be administered intravenously, it will be formulated in conventional vehicles, such as distilled water, saline, Ringer's solution or other conventional carriers.

It will be appreciated that preferred dosages of ACE inhibitor and AII antagonist as well as other antihypertensives disclosed herein will be as set out in the latest edition of the Physician's Desk Reference (PDR).

Other examples of preferred antihypertensive agents suitable for use herein include omapatrilat (Vanlev®) amlodipine besylate (Norvasc®), prazosin HCl (Minipress®), verapamil, nifedipine, nadolol, diltiazem, felodipine, nisoldipine, isradipine, nicardipine, atenolol, carvedilol, sotalol, terazosin, doxazosin, propranolol, and clonidine HCl (Catapres®).

Diuretics which may be employed in combination with compounds of formula I include hydrochlorothiazide, torasemide, furosemide, spironolactono, and indapamide.

Antiplatelet agents which may be employed in combination with Form N-1 of free acid I, Form N-2 of free acid I, and Form P-1 of the L-lysine salt of free acid I of the invention include aspirin, clopidogrel, ticlopidine, dipyridamole, prasugrel, abciximab, tirofiban, eptifibatide, anagrelide, and ifetroban, with or without aspirin, with clopidogrel and aspirin being preferred.

The antiplatelet drugs may be employed in amounts as indicated in the PDR. Ifetroban may be employed in amounts as set out in U.S. Pat. No. 5,100,889.

Antiosteoporosis agents suitable for use herein in combination with Form N-1 of free acid I, Form N-2 or free acid I or Form P-1 of the L-lysine salt of free acid I of the invention include parathyroid hormone or bisphosphonates, such as MK-217 (alendronate) (Fosamax®). Dosages employed will be as set out in the PDR.

In carrying our the method of the invention, a pharmaceutical composition will be employed containing Form N-1 of free acid I, Form N-2 or free acid I or Form P-1 of the L-lysine salt of free acid I, with or without another therapeutic agent, in association with a pharmaceutical vehicle or diluent for immediate release or extended release. The pharmaceutical composition can be formulated employing conventional solid or liquid vehicles or diluents and pharmaceutical additives of a type appropriate to the mode of desired administration. The compounds can be administered to mammalian species including humans, monkeys, dogs, etc. by an oral route, for example, in the form of tablets, capsules, granules, powders, suppositories, liposomes, inhalation sprays, or they can be administered by a parenteral route in the form of injectable preparations. The dose for adults is preferably between about 0.5 and 2,000 mg per day, preferably between about 1 to about 1000 mg/day, such as 1.5 mg per day, 2.5 mg per day, or 5 mg per day, which can be administered in a single dose or in the form of individual doses from 1-4 times per day.

Tablets are preferred. Most preferred are tablets containing the Form N-1 of the free acid I.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may also be orally delivered by sublingual and/or buccal administration, e.g. with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

It will be understood that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like. Thus, when the term “patient” is used herein, this term is intended to include all subjects, most preferably mammalian species.

A typical capsule for oral administration contains Form N-1 of free acid I, Form N-2 or free acid I or Form P-1 of the L-lysine salt of free acid I (2.5 mg), lactose (75 mg) and magnesium stearate (15 mg). The mixture is passed through a 60 mesh sieve and packed into a No. 1 gelatin capsule.

A typical injectable preparation is produced by aseptically placing 2.5 mg of Form N-1 of free acid I, Form N-2 or free acid I or Form P-1 of the L-lysine salt of free acid I into a vial, aseptically freeze-drying and sealing. For use, the contents of the vial are mixed with 2 mL of physiological saline, to produce an injectable preparation.

The following Examples represent preferred embodiments of the invention.

### ABBREVIATIONS

For ease of reference, the following abbreviations are employed herein, including the methods of preparation and Examples that follow:

### Ph=phenyl

### Bz=benzyl

t-Bu=tertiary butyl

### Me=methyl

### Et=ethyl

### Pr=propyl

### Iso-P=isopropyl

### MeOH=methanol

### EtOH=ethanol

### EtOAc=ethyl acetate

### Boc=tert-butyloxycarbonyl

### DCM=dichloromethane

### DCE=1,2-dichloroethane

### DMA=dimethyl acetal

### DMF=dimethyl formamide

### DMSO=dimethyl sulfoxide

### IPA=isopropyl alcohol

### THF=tetrahydrofuran

### TFA=trifluoroacetic acid

### KOH=potassium hydroxide

### K₂CO₃=potassium carbonate

### NaOH=sodium hydroxide

min=minute(s)

### L=liter

ml or mL=milliliter

### μL=microliter

g=gram(s)
mg=milligram(s)
mol=moles
mmol=millimole(s)
meq=milliequivalent

### RT or rt=room temperature (20 to 25° C.)

ret. t.=HPLC retention time (minutes)
sat or sat'd=saturated
aq.=aqueous

### TLC=thin layer chromatography

### HPLC=high performance liquid chromatography

### RP HPLC=reverse phase HPLC

### LC/MS=high performance liquid chromatography/mass spectrometry

### MS=mass spectrometry

### NMR=nuclear magnetic resonance

mp=melting point

### RH=relative humidity

In the Examples, designations associated with HPLC data reflect the following conditions:

a. Column: YMC ODSA s-5 5u C18 4.6×50 mm; Solvent: solvent A=10% MeOH/90% water/0.1% THF, and solvent B=90% MeOH/10% water/0.1% THF; Method: 4 min gradient;

b. Column: YMC s5 ODS 4.6×50 mm; Solvent: solvent A=10% MeOH/90% water/0.2% H₃PO₄, and solvent B=90% MeOH/10% water/0.2% H₃PO₄; Method: 4 min gradient.

### EXAMPLES

The invention will now be further described by the following working examples, which are preferred embodiments of the invention. HPLC purifications were done on C18 reverse phase (RP) columns using water MeOH mixtures and TFA as buffer solution. These examples are illustrative rather than limiting. There may be other embodiments that fall within the spirit and scope of the invention as defined by the appended claims.

The following Examples represent preferred embodiments of the invention.

### Example 1

### Preparation of Form N-1 Crystals of Free Acid I (Peliglitazar)

Crude peliglitazar (free acid I) prepared as described in U.S. Pat. No. 6,653,314 (Example 498A) was crystallized in isopropyl alcohol to form crystals of Form N-1 as follows.

Crude peliglitazar (free acid I) prepared as described in U.S. Pat. No. 6,653,314 (Example 498A) (1 g) was dissolved in 6 ml of isopropyl alcohol at about 45° C. to obtain a clear solution.

The above solution was seeded with crystalline seeds of Form N-1 (prepared by recrystallizing or slurrying the crude free acid I in hexane (as described in Example 1A) with stirring to initiate crystallization and the seeded solution was cooled to 20° C. over 2 hours. Crystallization started with formation of a white crystal slurry which became gradually thicker. The slurry was filtered at 20° C. and the resulting wet cake was washed with cold isopropyl alcohol and dried in vacuo at 35° C. for 48 hours. A white crystalline power was obtained at 88% yield. The product obtained was the crystalline Form N-1 of the free base I.

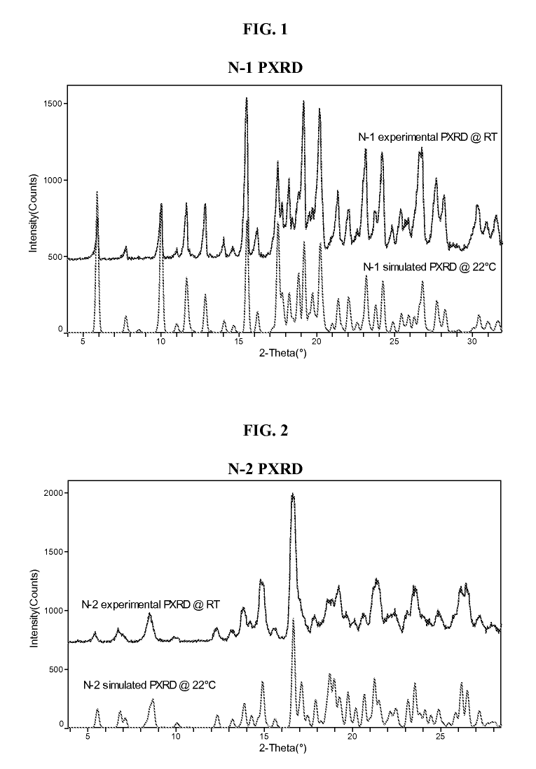

Calculated and observed powder X-ray diffraction patterns (PXRD) of crystals of Example 1 Form N-1 free base I are shown in FIG. 1.

The diffractogram of crystals of the Example 1 Form N-1 of the free base I shown in FIG. 1 exhibits 2θ values at a temperature of 22° C. of (CuKα λ=1.5418 Å) of 5.9±0.1, 77±0.1, 10.0±0.1, 11.7±0.1, 12.8±0.1, 15.5±0.1, 16.2±0.1, 17.5±0.1, 19.2±0.1, 20.2±0.1.

Unit Cell Data is shown in Table 2.

A differential scanning calorimetry (DSC) thermogram of crystals of Example 1 Form N-1 of free base I is shown in FIG. 4.

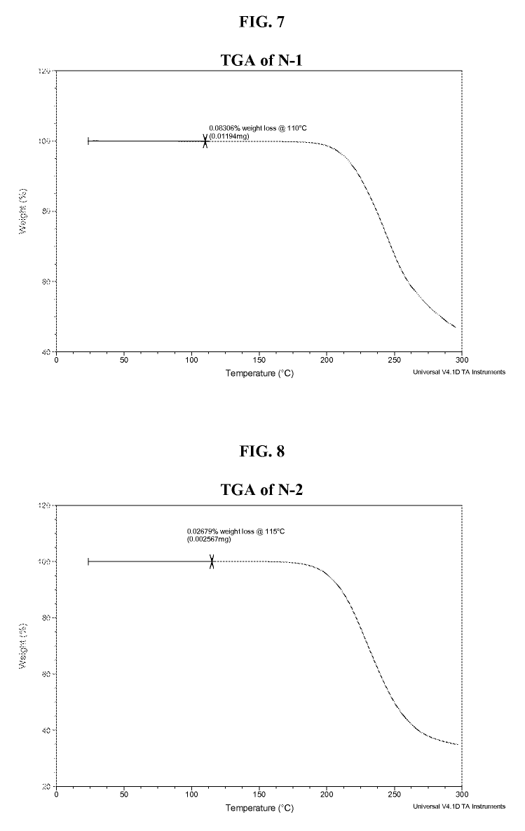

A thermogravimetric analysis (TGA) curve of Example 1 Form N-1 of free base I is shown in FIG. 7.

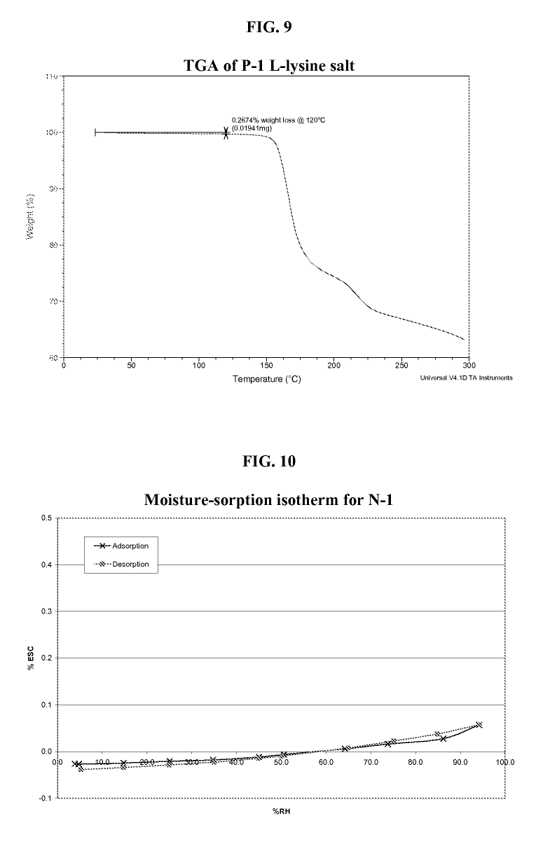

A moisture-sorption isotherm of Form N-1 crystals of free base I is shown in FIG. 10.

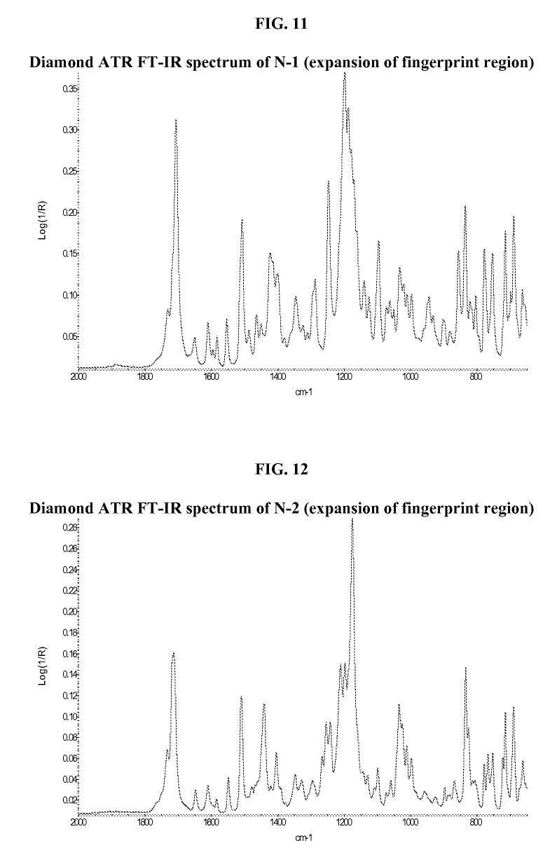

A Diamond ATR FT-IR spectrum of Form N-1 crystals of free base I is shown in FIG. 11 which exhibits the following selected unique IR vibrational bands (cm⁻¹): 1707, 1554, 1247, 1051, 856.

### Example 1A

### Preparation of Seed Crystals of Free Acid I (Form N-1)

The seed crystals of free acid I were prepared by slurring or crystallizing the amorphous form (as described in U.S. Pat. No. 6,653,314 B2 (Example 498A)) in hexane. Approximately 20 mg of the amorphous form of free acid I was placed in a glass vial, to which 75 microliters of hexane was added to form a slurry. The vial was closed and stored at room temperature without agitation. Long rod-shaped crystals were observed in the slurry after three days. The crystals showed very strong birefrigency and was confirmed to be Form N-1 by single crystal structure analysis, PXRD, Hot Stage Microscopy, TGA and DSC.

The seed crystals can also be obtained from heptane or hexane/ethyl acetate under similar conditions. The crystals obtained from hexane/ethyl acetate (5:1) were irregular plates in morphology.

### Example 2

### Alternative Preparation of Form N-1 Crystals of Free Acid I (Peliglitazar)

Crude peliglitazar (free acid I) prepared as described in U.S. Pat. No. 6,653,314 (Example 498A) was crystallized in anhydrous ethanol to form crystals of Form N-1 as follows.

Crude peliglitazar (free acid I) (67 mg) was dissolved in 0.5 ml of anhydrous ethanol at about 45° C. to obtain a clear solution.

The above solution was seeded with crystalline seeds of Form N-1 (prepared by recrystallizing or slurrying the crude free acid I in hexane (as described in Example 1A) with stirring to initiate crystallization and the seeded solution was cooled to 20° C. over 2 hours. Crystallization started resulting in formation of a white crystal slurry which became gradually thicker. The slurry was filtered at 20° C. and the resulting wet cake was washed with cold isopropyl alcohol and dried in vacuo at 35° C. for 10 hours. A white crystalline power was obtained at 67% yield. The product obtained was the crystalline Form N-1 of the free base I.

### Example 3

### Preparation of Form N-2 Crystals of Free Acid I (Peliglitazar)

Crude peliglitazar (free acid I) prepared as described in U.S. Pat. No. 6,653,314 (Example 498A) was crystallized in isopropyl alcohol to form crystals of Form N-2 as follows.

Crude peliglitazar (free acid I) (0.5 g) was dissolved in 5 ml of isopropyl alcohol at about 55° C. to form a solution.

The above solution was seeded with crystalline seeds of Form N-2 (prepared by crystallizing or slurrying crude free acid I in IPA) with stirring to initiate crystallization and the seeded solution was cooled to 35° C. over 8 hours. The resulting slurry was cooled to 20° C. and filtered. The resulting wet cake was washed with cold isopropyl alcohol and dried in vacuo at 35° C. to obtain a white crystalline powder which is crystalline Form N-2 of Free Acid I.

Calculated and observed powder X-ray diffraction powders (PXRD) of the Example 3 Form N-2 free base I are shown in FIG. 2.

The diffractogram of crystals of the Example 3 Form N-2 of the free base I shown in FIG. 2 exhibits 2θ values at a temperature of 22° C. of (CuKα λ=1.5418 Å) 3.6±0.1, 5.6±0.1, 6.8±0.1, 8.7±0.1, 12.3±0.1, 13.9±0.1, 14.9±0.1, 16.7±0.1, 17.1±0.1, 23.5±0.1.

Unit Cell Data is shown in Table 2.

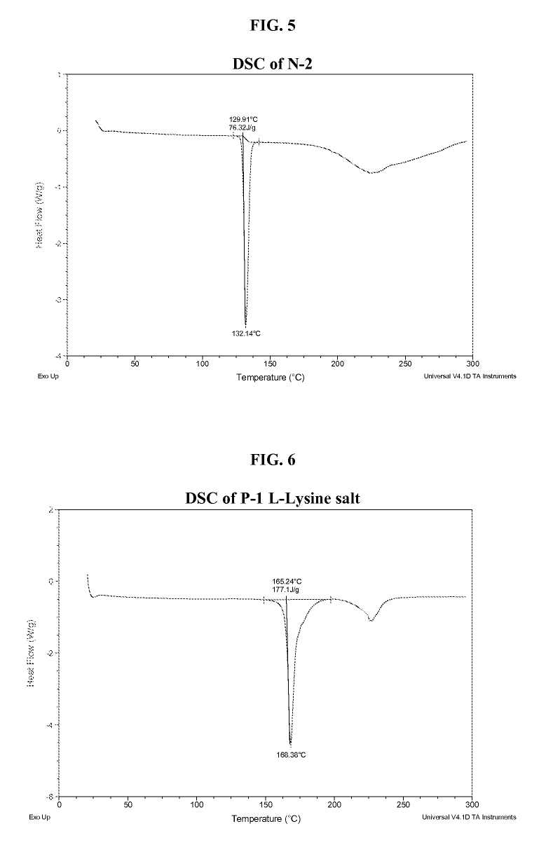

A differential scanning calorimetry (DSC) thermogram of Example 3 Form N-2 of free base I is shown in FIG. 5.

A thermogravimetric analysis (TGA) curve of Example 3 Form N-2 of free base I is shown in FIG. 8.

A Diamond ATR FT-IR spectrum of Form N-2 crystals of free base I is shown in FIG. 12 which exhibits the following selected unique IR vibrational bands (cm⁻¹): 1713, 1548, 1267, 1254, 1242, 825, 767.

### Example 4

### Preparation of Form P-1 Crystals of Lysine Salt of Free Acid I (Peliglitazar)

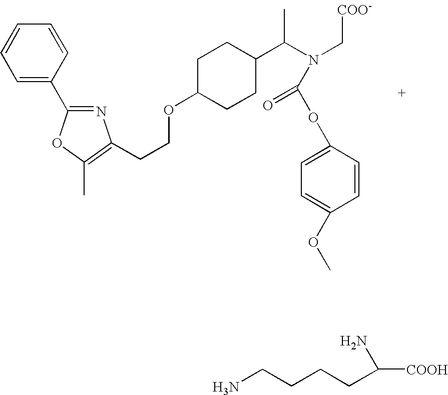

0.2 Crude peliglitazar (free acid I) prepared as described in U.S. Pat. No. 6,653,314 (Example 498A) was dissolved in 10 ml anhydrous ethanol at 59° C. 0.056 g of L-lysine was added to the solution of free acid I in anhydrous ethanol and the mixture was stirred vigorously at 59° C. The L-lysine particles slowly dissolved and a white crystal slurry was obtained after 1 hour. The crystal slurry was cooled to 20° C. over 2 hours and filtered. The resulting wet cake was washed with cold ethanol and dried in vacuo at 35° for 15 hours to obtain a white crystalline powder at 85% yield. The product obtained was the crystalline Form P-1 of the L-lysine salt of free base I.

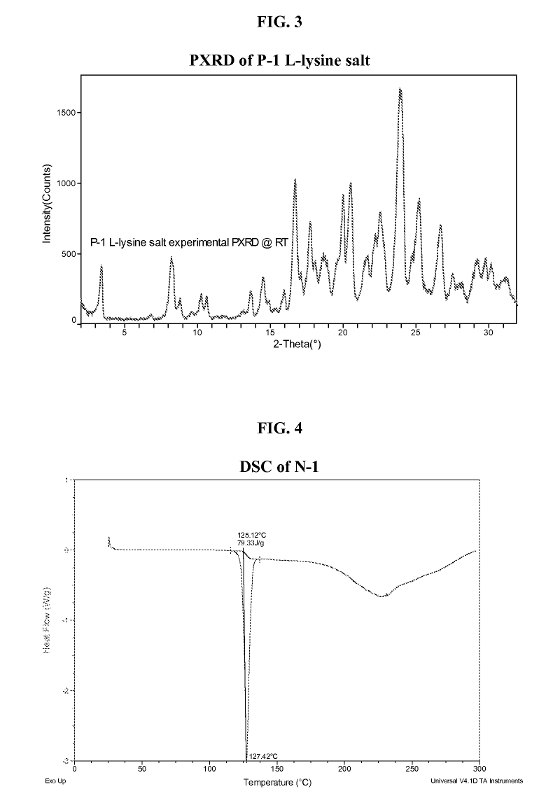

Calculated and observed powder X-ray diffraction patterns of the Example 4 Form P-1 of L-lysine salt of free base I are shown in FIG. 3.

Form P-1 of the L-lysine salt of the free base I exhibits 2θ values at a temperature of 22° C. (CuKα λ=1.5418 Å) of 3.4±0.1, 8.2±0.1, 13.6±0.1, 14.57±0.1, 16.7±0.1, 20.0±0.1, 20.5±0.1, 22.5±0.1, 23.9±0.1, 25.1±0.1, 26.7±0.1.

A differential scanning calorimetry thermogram (DSC) of Example 4 Form P-1 L-lysine salt of free base I is shown in FIG. 6.

A thermogravimetric analysis (TGA) curve of Example 4 Form P-1 L-lysine salt of free base I is shown in FIG. 9.

### Example 5

### Alternative Preparation of Form P-1 Crystals of Lysine Salt of Free Acid I (Peliglitazar)

0.2 Crude peliglitazar (free acid I) prepared as described in U.S. Pat. No. 6,653,314 (Example 498A) was dissolved in 10 ml anhydrous ethanol at 40° C. 0.055 g of L-lysine was added to the solution of free acid I in anhydrous ethanol and the mixture was stirred vigorously at 40° C. The L-lysine particles slowly dissolved and a thick white crystal slurry was obtained after 1 hour. The crystal slurry was cooled to 20° C. and filtered. The resulting wet cake was washed with cold ethanol and dried in vacuo at 35° for 15 hours to obtain a white crystalline powder at 76%. The product obtained was the crystalline Form P-1 of the L-lysine salt of free base I.

### Example 6

### Studies of Crystal Forms Prepared in Previous Examples

X-ray powder diffraction (PXRD) data were obtained using a Bruker C2 GADDS (General Area Detector Diffraction System). The radiation was Cu Kα (40 KV, 50 mA). The sample-detector distance was 15 cm. Powder samples were placed in sealed glass capillaries of 1 mm or less in diameter; the capillary was rotated during data collection. Data were collected for 3≦2θ≦35° with a sample exposure time of at least 2000 seconds. The resulting two-dimensional diffraction arcs were integrated to create a traditional 1-dimensional PXRD pattern with a step size of 0.02 degrees 2θ in the range of 3 to 35 degrees 2θ.

Single crystal X-ray data were collected on a Bruker-Nonius CAD4 serial diffractometer (Bruker Axs, Inc., Madison Wis.). Unit cell parameters were obtained through least-squares analysis of the experimental diffractometer settings of 25 high-angle reflections. Intensities were measured using Cu Kα radiation (λ=1.5418 Å) at a constant temperature with the θ-2θ variable scan technique and were corrected only for Lorentz-polarization factors. Background counts were collected at the extremes of the scan for half of the time of the scan. Alternately, single crystal data were collected on a Bruker-Nonius Kappa CCD 2000 system using Cu Kα radiation (λ=1.5418 Å). Indexing and processing of the measured intensity data were carried out with the HKL2000 software package in the Collect program suite R. Hooft, Nonius B. V. (1998). When indicated, crystals were cooled in the cold stream of an Oxford cryogenic system during data collection.

The structures were solved by direct methods and refined on the basis of observed reflections using either the SDP software package SDP, Structure Determination Package, Enraf-Nonius, Bohemia, N.Y.) with minor local modifications or the crystallographic package, MAXUS (maXus solution and refinement software suit: S. Mackay, C. J. Gilmore, C. Edwards, M. Tremayne, N. Stewart, and K. Shankland. maXus is a computer program for the solution and refinement of crystal structures from diffraction data.

The derived atomic parameters (coordinates and temperature factors) were refined through full matrix least-squares. The function minimized in the refinements was Σ_{w}(|Fₒ|−|F_{c}|)². R is defined as Σ∥F|−|F∥/Σ|Fₒ| while R_{w}=[Σ_{w}(|Fₒ|−|F_{c}|)²/Σ_{w}|Fₒ|^{2]}^{1/2} where w is an appropriate weighting function based on errors in the observed intensities. Difference maps were examined at all stages of refinement. Hydrogen atoms were introduced in idealized positions with isotropic temperature factors, but no hydrogen parameters were varied.

“Hybrid” simulated powder X-ray patterns were generated as described in the literature (Yin. S.; Scaringe, R. P.; DiMarco, J.; Galella, M. and Gougoutas, J. Z., American Pharmaceutical Review (2003), 6(2), 80). The room temperature cell parameters were obtained by performing a cell refinement using the CellRefine.xls program. Input to the program includes the 2-theta position of ca. 10 reflections, obtained from the experimental room temperature powder pattern; the corresponding Miller indices, hkl, were assigned based on the single-crystal data collected at low temperature. A new (hybrid) PXRD was calculated (by either of the software programs, Alex or LatticeView) by inserting the molecular structure determined at low temperature into the room temperature cell obtained in the first step of the procedure. The molecules are inserted in a manner that retains the size and shape of the molecule and the position of the molecules with respect to the cell origin, but, allows intermolecular distances to expand with the cell.

The characteristic diffraction peak positions (degrees 2θ±0.1) at RT of PXRD patterns shown in the accompanying Figures are based on high quality patterns collected with a diffractometer (CuKα) with a spinning capillary with 2θ calibrated with an NIS or other suitable standard.

Hot stage microscopy (LVL)—Crystals were placed on a glass slide, covered with a cover slip, and heated on a Linkam LTS350 hot stage mounted on a microscope. The heating rate was controlled at 10°/min for the temperature range, ambient to 300° C. The crystals were observed visually for evidence of phase transformation, changes in birefringence, opacity, and melting.

Differential scanning calorimetry (DSC) experiments were performed in a TA Instruments™ model Q1000. The sample (about 2-6 mg) was weighed in an aluminum pan and recorded accurately recorded to a hundredth of a milligram, and transferred to the DSC. The instrument was purged with nitrogen gas at 50 mL/min. Data were collected between room temperature and 300° C. at 10° C./min heating rate. The plot was made with the endothermic peaks pointing down.

Thermal gravimetric analysis (TGA) experiments were performed in a TA Instruments™ model Q500. The sample (about 10-30 mg) was placed in a platinum pan previously tared. The weight of the sample was measured accurately and recorded to a thousand of a milligram by the instrument. The furnace was purged with nitrogen gas at 100 mL/min. Data were collected between room temperature and 300° C. at 10° C./min heating rate.

Moisture sorption isotherms were collected in a VTI SGA-100 Symmetric Vapor Analyzer using approximately 10 mg of sample. The sample was dried at 60° C. until the loss rate of 0.0005 wt %/min was obtained for 10 minutes. The sample was tested at 25° C. and 3 or 4, 5, 15, 25, 35, 45, 50, 65, 75, 85, and 95% RH. Equilibration at each RH was reached when the rate of 0.0003 wt %/min for 35 minutes was achieved or a maximum of 600 minutes.

The various crystalline forms of free acid I were prepared and are tabulated in Table 1. The unit cell data and other properties for all crystalline forms of the invention are tabulated and summarized in Table 2. The unit cell parameters were obtained from single crystal X-ray crystallographic analysis. A detailed account of unit cells can be found in Chapter 3 of Stout & Jensen, “X-Ray Structure Determination: A Practical Guide”, (MacMillian, 1968).

TABLE 1

| Form | Description |
|---|---|
| N-1 | Neat crystal |
| N-2 | Neat crystal |
| P-1 L-lysine salt | Neat crystal |

TABLE 2
Unit Cell Data

| Form | T | a(Å) | b(Å) | c(Å) | α° | β° | γ° | Z′ | SG | Vₘ | R | D_{calc} |
|---|---|---|---|---|---|---|---|---|---|---|---|---|
| N-1 | +22 | 10.387(3) | 17.638(2) | 15.073(4) | 90 | 96.70(2) | 90 | 2 | P2₁ | 686 | .04 | 1.285 |
| N-2 | +22 | 5.346(1) | 20.75(2) | 49.59(4) | 90 | 90 | 90 | 2 | orthorhombic | 687 | | 1.281 |

T = temp (° C.) for the crystallographic data.
Z′ = number of drug molecules per asymmetric unit
Vₘ = V(unit cell)/(Z drug molecules per cell)
R = residual index
D_{calc} = density of crystal calculated
SG = space group

TABLE 3
Table of Positional Parameters and Their Estimated
Standard Deviations for Form N-1

| Atom | x | y | z | B(iso) |
|---|---|---|---|---|
| 01 | −0.025(1) | −0.0293(3) | 0.7409(3) | 4.9(2) |
| O1A | −0.2361(6) | −0.0139(4) | 0.6945(4) | 6.5(2) |
| O11 | 0.1116(6) | 0.2246(4) | 0.2704(4) | 5.2(2) |
| O17 | 0.1278(7) | 0.3352(4) | −0.0081(4) | 6.4(2) |
| O26A | −0.1735(6) | −0.1081(3) | 0.5100(4) | 4.9(2) |
| O26 | −0.3177(5) | −0.0395(3) | 0.4183(4) | 4.3(1) |
| O33 | −0.4502(8) | −0.2764(4) | 0.1853(4) | 6.8(2) |
| N3 | −0.1842(7) | 0.0209(4) | 0.5241(4) | 3.3(2) |
| N-15 | 0.0979(7) | 0.3846(4) | 0.1233(4) | 4.0(2) |
| C1 | −0.1216(8) | −0.0101(5) | 0.6828(5) | 4.0(2) |
| C2 | −0.0787(8) | 0.0182(5) | 0.5961(5) | 3.7(2) |
| C4A | −0.2760(9) | 0.1428(5) | 0.5673(6) | 4.3(2) |
| C4 | −0.2393(9) | 0.0951(5) | 0.4892(6) | 4.1(2) |
| C5 | −0.1443(8) | 0.1334(5) | 0.4342(5) | 3.2(2) |
| C6 | −0.1100(9) | 0.0966(5) | 0.3579(5) | 4.0(2) |
| C7 | −0.027(1) | 0.1285(5) | 0.3064(6) | 5.3(3) |
| C8 | 0.0268(9) | 0.1980(5) | 0.3255(5) | 4.0(2) |
| C9 | −0.0032(9) | 0.2377(5) | 0.4017(6) | 4.2(2) |
| C10 | −0.0896(9) | 0.2028(5) | 0.4539(5) | 3.5(2) |
| C12 | 0.1758(9) | 0.2961(5) | 0.2946(6) | 4.6(3) |
| C13 | 0.2665(9) | 0.3102(6) | 0.2261(6) | 4.8(3) |
| C14 | 0.1950(9) | 0.3289(5) | 0.1353(6) | 4.4(2) |
| C16 | 0.0612(9) | 0.3853(5) | 0.0371(5) | 4.4(2) |
| C18 | 0.217(1) | 0.2992(5) | 0.0564(6) | 5.6(3) |
| C19 | 0.303(1) | 0.2397(7) | 0.0263(8) | 9.0(4) |
| C20 | −0.036(1) | 0.4337(5) | −0.0111(5) | 4.7(2) |
| C21 | −0.117(1) | 0.4772(6) | 0.0349(6) | 6.3(3) |
| C22 | −0.206(1) | 0.5234(7) | −0.0111(7) | 7.7(3) |
| C23 | −0.212(1) | 0.5286(7) | −0.1031(7) | 9.5(4) |
| C24 | −0.134(1) | 0.4844(6) | −0.1481(6) | 8.1(4) |
| C25 | −0.041(1) | 0.4363(6) | −0.1038(6) | 6.7(3) |
| C26 | −0.2173(8) | −0.0464(5) | 0.4872(5) | 3.5(2) |
| C27 | −0.3473(9) | −0.1027(5) | 0.3641(5) | 4.0(2) |
| C28 | −0.330(1) | −0.0972(5) | 0.2764(6) | 5.4(3) |
| C29 | −0.366(1) | −0.1560(5) | 0.2168(6) | 5.7(3) |
| C30 | −0.4189(9) | −0.2216(5) | 0.2489(6) | 4.7(2) |
| C31 | −0.4371(9) | −0.2264(5) | 0.3376(6) | 4.8(3) |
| C32 | −0.399(1) | −0.1666(5) | 0.3946(6) | 5.6(3) |
| C33 | −0.491(1) | −0.3469(6) | 0.2164(8) | 9.1(4) |
| H11 | −0.063 | −0.048 | 0.802 | 6.1 |
| O41 | 0.4766(6) | −0.0308(4) | 0.7058(3) | 5.4(2) |
| O41A | 0.2702(6) | −0.0361(5) | 0.6500(4) | 6.7(2) |
| O51 | 0.6236(6) | 0.2192(4) | 0.2414(4) | 5.1(2) |
| O57 | 0.5484(6) | 0.3847(4) | −0.0074(4) | 5.0(2) |
| O66 | 0.1937(6) | −0.0756(3) | 0.3784(4) | 5.4(2) |
| O66A | 0.3231(6) | −0.1424(3) | 0.4805(4) | 5.0(2) |
| O73 | −0.0143(8) | −0.3337(4) | 0.2086(5) | 8.0(2) |
| N43 | 0.3343(7) | −0.0141(4) | 0.4772(4) | 4.2(2) |
| N55 | 0.5678(7) | 0.4137(4) | 0.1360(4) | 3.7(2) |
| C41 | 0.3814(8) | −0.0281(6) | 0.6407(6) | 4.4(2) |
| C42 | 0.4354(8) | −0.0155(5) | 0.5521(5) | 4.3(2) |
| C44 | 0.2809(8) | 0.0613(5) | 0.4423(5) | 3.4(2) |
| C45 | 0.3804(8) | 0.1015(5) | 0.3946(5) | 3.5(2) |
| C46 | 0.4231(9) | 0.0736(5) | 0.3188(5) | 4.1(2) |
| C47 | 0.5034(9) | 0.1122(5) | 0.2686(5) | 4.4(2) |
| C48 | 0.5459(9) | 0.1846(5) | 0.2971(6) | 3.9(2) |
| C49 | 0.511(1) | 0.2114(5) | 0.3761(5) | 4.6(3) |
| C50 | 0.4283(9) | 0.1720(5) | 0.4238(5) | 4.2(2) |
| C51 | 0.2305(9) | 0.1039(6) | 0.5173(6) | 5.0(3) |
| C52 | 0.6781(9) | 0.2919(6) | 0.2725(5) | 4.8(2) |
| C53 | 0.7476(9) | 0.3241(6) | 0.1985(6) | 5.2(3) |
| C54 | 0.6557(9) | 0.3555(5) | 0.1211(6) | 4.6(2) |
| C56 | 0.5047(8) | 0.4283(5) | 0.0581(5) | 3.8(2) |
| C58 | 0.646(1) | 0.3390(5) | 0.0345(6) | 5.1(3) |
| C59 | 0.708(1) | 0.2866(6) | −0.0231(7) | 6.8(3) |
| C60 | 0.4033(9) | 0.4829(5) | 0.0333(5) | 4.3(2) |
| C61 | 0.3273(8) | 0.5062(5) | 0.0971(6) | 4.0(2) |
| C62 | 0.2258(9) | 0.5568(6) | 0.0737(7) | 5.0(3) |
| C63 | 0.202(1) | 0.5815(5) | −0.0153(7) | 6.3(3) |
| C64 | 0.278(1) | 0.5562(6) | −0.0764(7) | 6.2(3) |
| C65 | 0.380(1) | 0.5079(5) | −0.0550(6) | 6.1(3) |
| C66 | 0.2884(9) | −0.0808(5) | 0.4499(6) | 4.7(2) |
| C67 | 0.142(1) | −0.1431(5) | 0.3385(6) | 4.9(3) |
| C68 | 0.2097(9) | −0.1828(6) | 0.2814(6) | 5.4(3) |
| C69 | 0.153(1) | −0.2454(6) | 0.2368(6) | 5.4(3) |
| C70 | 0.032(1) | −0.2667(6) | 0.2539(6) | 5.7(3) |
| C71 | −0.0353(9) | −0.2281(6) | 0.3112(6) | 5.7(3) |
| C72 | 0.021(1) | −0.1643(6) | 0.3535(6) | 5.1(3) |
| C73 | −0.137(1) | −0.3588(7) | 0.2184(9) | 9.2(4) |
| H441 | 0.196 | 0.050 | 0.393 | 4.6 |

Table 4 sets out characteristic diffraction peak positions for Forms N-1 and N-2.

TABLE 4
Characteristic diffraction peak positions (degrees
2θ ± 0.1) @ RT, based on a high quality
pattern collected with a diffractometer (CuKα) with
a spinning capillary with 2θ calibrated with
a NIST other suitable standard

| N-1 | N-2 |
|---|---|
| 5.9 | 3.6 |
| 7.7 | 5.6 |
| 10.0 | 6.8 |
| 11.7 | 8.7 |
| 12.8 | 12.3 |
| 15.5 | 13.9 |
| 16.2 | 14.9 |
| 17.5 | 16.7 |
| 19.2 | 17.1 |
| 20.2 | 23.5 |

Table 5 sets out selected unique IR vibrational bands for Forms N-1 and N-2.

TABLE 5
Selected unique IR vibrational bands (cm⁻¹) for N-1 and N-2

| N-1 | N-2 |
|---|---|
| 1707 | 1713 |
| 1554 | 1548 |
| 1247 | 1267 |
| 1051 | 1254 |
| 856 | 1242 |
| | 825 |
| | 767 |

What is claimed is:

1. A crystalline form of the free acid I which has the structure 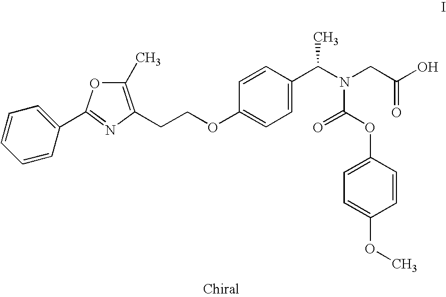
(also referred to as peliglitazar)
or a salt thereof.

2. The crystalline form as defined in claim 1 which is Form N-1 of the free acid I, Form N-2 of the free acid I or Form P-1 of the L-lysine salt of the free acid I.

3. Form N-1 of crystalline free acid I.

4. The crystalline form as defined in claim 3 which is Form N-1 of the free acid I and is characterized by unit cell parameters substantially equal to the following:
Cell Dimensions:
a=10.387(3) Å
b=17.638(2) Å
c=15.073(4) Å
α=90°
β=96.70(2)
γ7=90°
Space group P2₁
Molecules/asymmetric unit 2
wherein said crystalline form is at about +22° C.

5. The crystalline form as defined in claim 3 which is Form N-1 of the free acid I and is characterized by fractional atomic coordinates substantially as listed in Table 3.

6. The crystalline form as defined in claim 3 which is Form N-1 of the free acid I and is characterized by a powder X-ray diffraction pattern substantially in accordance with that shown in FIG. 1.

7. The crystalline form as defined in claim 3 which is Form N-1 of the free acid I having a powder X-ray diffraction pattern comprising the following 2θ values at a temperature of 22° C. (CuKα λ=1.5418 Å) 5.9±0.1, 7.7±0.1, 10.1±0.1, 11.7±0.1, 12.8±0.1, 15.5±0.1, 16.2±0.1, 17.5±0.1, 19.2±0.1, and 20.2±0.1.

8. The crystalline form as defined in claim 3 which is Form N-1 of the free acid I and is characterized by a differential scanning calorimetry thermogram substantially in accordance with that shown in FIG. 4, having an endotherm with peak onset in the range from about 123 to about 128° C.

9. The crystalline form as defined in claim 3 which is Form N-1 of the free acid I and is characterized by a thermal gravimetric analysis curve substantially in accordance with that shown in FIG. 7 having a negligible weight loss up to about 110° C.

10. The crystalline form as defined in claim 3 which is Form N-1 of the free acid I and is characterized by a moisture-sorption isotherm substantially in accordance with that shown in FIG. 10 having negligible moisture uptake in the range from 25 to 75% RH at 25° C.

11. The crystalline form as defined in claim 3 which is Form N-1 of the free acid I having a Diamond ATR FT-IR spectrum substantially as shown in FIG. 11.

12. The crystalline form as defined in claim 3 which is Form N-1 of the free acid I having IR vibrational bands (cm⁻¹) at 1707, 1554, 1247, 1051, and 856.

13. The crystalline form as defined in claim 3 which is Form N-1 of the free acid I having a melting point between about 122 and 128° C.

14. Form N-2 of the crystalline free acid I.

15. The crystalline form as defined in claim 14 which is Form N-2 of the free base I and is characterized by unit cell parameters substantially equal to the following:
Cell Dimensions:
a=5.346(1) Å
b=20.75(2) Å
c=49.59(4) Å
α=90°
β=90°
γ=90°
Space group/orthorhombic
Molecules/asymmetric unit 2
wherein said crystalline form is at about room temperature.

16. The crystalline form as defined in claim 14 which is Form N-2 of the free base I as characterized by a powder X-ray diffraction pattern substantially in accordance with that shown in FIG. 2.

17. The crystalline form as defined in claim 14 which is Form N-2 of the free base I as characterized by a powder X-ray diffraction pattern comprising the following 2θ values at a temperature of 22° C. (CuKα λ-1.5418 Å) of 3.6±0.1, 5.6±0.1, 6.8÷0.1, 8.7±0.1, 12.3±0.1, 13.9±0.1, 14.9±0.1, 16.7±0.1, 17.1±0.1, and 23.5±0.1 at about room temperature.

18. The crystalline form as defined in claim 14 which is Form N-2 of the free acid I which is characterized by Diamond ATR FT-IR spectrum substantially as shown in FIG. 12.

19. The crystalline form as defined in claim 14 which is Form N-2 of the free acid I which is characterized by IR vibrational bands (cm⁻¹) at 1713, 1548, 1267, 1254, 1242, 825, and 767.

20. The crystalline form as defined in claim 14 which is Form N-2 of free acid I and is characterized by a differential scanning calorimetry thermogram substantially in accordance with that shown in FIG. 5, having an endotherm with peak onset at about 130°.

21. The crystalline form as defined in claim 14 which is Form N-2 of free acid I and is characterized by a thermal gravimetric analysis curve in accordance with that shown in FIG. 8 having a negligible weight loss up to about 115° C.

22. Form P-1 of the lysine salt of the free acid I of the structure

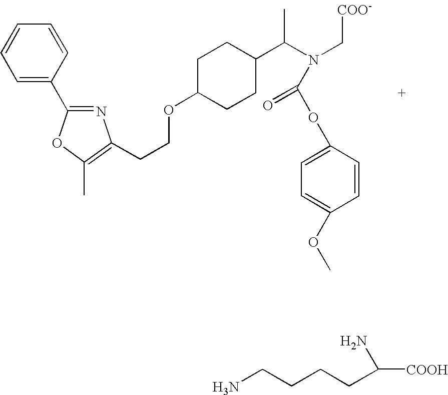

23. The crystalline form as defined in claim 22 which is Form P-1 of the L-lysine salt of the free acid I characterized by a powder X-ray diffraction pattern substantially in accordance with that shown in FIG. 3.

24. The crystalline form as defined in claim 22 which is Form P-1 of the L-lysine salt of the free acid I characterized by a powder X-ray diffraction pattern comprising the following 2θ values at a temperature of 22° C. (CuKα λ=1.5418 Å) of 3.4±0.1, 8.2±0.1, 13.6±0.1, 14.57±0.1, 16.7±0.1, 20.0±0.1, 20.5±0.1, 22.5±0.1, 23.9±0.1, 25.1±0.1, 26.7±0.1.

25. The crystalline form as defined in claim 22 which is characterized by a differential scanning calorimetry thermogram substantially in accordance with that shown in FIG. 6, having an endotherm with peak onset at about 165° C.

26. The crystalline form as defined in claim 22 which is characterized by a thermal gravimetric analysis curve in accordance with that shown in FIG. 9 having a weight loss of about 0.3% up to about 120° C.

27. A process for preparing the crystalline Form N-1 of free acid I, which comprises
a) providing the free acid I having the structure 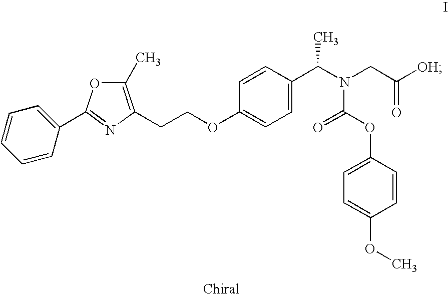
b) dissolving the free acid I in an organic solvent;
c) seeding the solution from step b) with crystalline seeds of Form N-1 of the free acid I to initiate crystallization, and form a slurry; and
d) recovering crystalline Form N-1 of the free acid I.

28. The process as defined in claim 27 including the step of preparing the seed crystals of Form N-1 of the free acid I by recrystallizing or slurrying the crude free acid I in hexane, heptane or hexane-ethyl acetate mixture.

29. The process as defined in claim 1 wherein the free acid I is dissolved in methanol, ethanol, toluene, isopropyl alcohol, methanol/water, acetonitrile/water, N,N-dimethylacetamide (DMA), acetone, 2-butanone (MEK) or butyl acetate.

30. The process as defined in claim 1 wherein the free acid I is dissolved in ethanol or isopropyl alcohol.

31. The process as defined in claim 1 wherein the solution from step b) seeded with seeds of Form N-1 of free acid I is cooled to a temperature within the range from about 5 to about 25° C.

32. A process for preparing the crystalline Form N-1 of free acid I, which comprises
a) providing free acid I;
b) dissolving the free acid I in isopropyl alcohol;
c) seeding the solution from step b) with crystalline seeds of Form N-2 of free acid I to initiate crystallization; and
d) recovering crystalline Form N-2 of the free acid I.

33. A process is for preparing the crystalline L-lysine salt of free acid I, which comprises
a) providing free acid I;
b) dissolving the free acid I in ethanol to obtain a solution;
c) admixing L-lysine with the solution from step b) to form a slurry;
d) cooling the slurry to a temperature within the range from about 5 to about 25° C.; and
e) recovering the crystalline L-lysine salt of free acid I.

34. The process as defined in claim 33 wherein the mixture of L-lysine and the solution from step b) is heated at a temperature within the range from about 35 to about 65° C.

35. The process as defined in claim 33 including the step of cooling the slurry from step c) at a temperature from about 5 to about 25° C.

36. Form N-1 of the free acid I 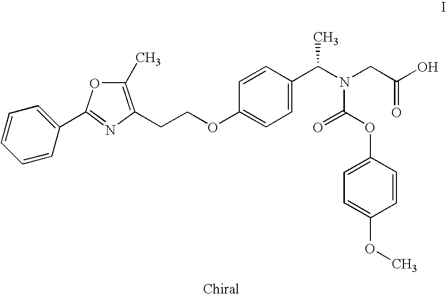
prepared by the process defined in claim 27.

37. Form N-2 of the free acid 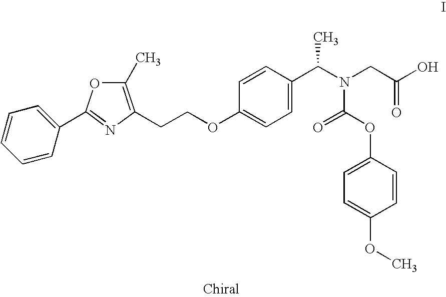
prepared as defined by the process of claim 32.

38. Form P-1 of the L-lysine salt of the free acid I 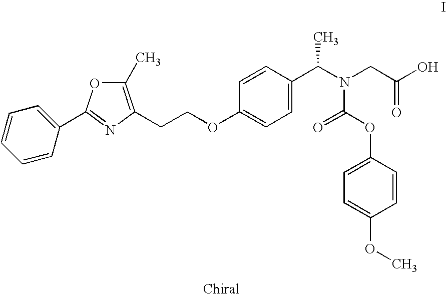
prepared as defined by the process of claim 33.

39. A pharmaceutical composition comprising a compound according to claim 1 and a pharmaceutically-acceptable carrier or diluent.

40. A pharmaceutical composition comprising a compound according to claim 2 and a pharmaceutically-acceptable carrier or diluent.

41. A pharmaceutical composition comprising a compound according to claim 3 and a pharmaceutically acceptable carrier or diluent.

42. A pharmaceutical composition comprising a compound as defined in claim 22 and a pharmaceutically acceptable carrier or diluent.

43. A method of treating diabetes, dyslipidemia or atherosclerosis which comprises administering to a human patient in need of such treatment a therapeutic amount of a pharmaceutical composition according to claim 40.
